(19) United States
(12) Patent Application Publication
(10) Pub. No.: US 2013/0045988 A1
(43) Pub. Date: Feb. 21, 2013

(54) COMBINATION THERAPY

(71) Applicants: Romain Adelin Lefebvre, Drongen (BE); Joris Herman De Maeyer, Mechelen (BE)

(72) Inventors: Romain Adelin Lefebvre, Drongen (BE); Joris Herman De Maeyer, Mechelen (BE)

(73) Assignee: SHIRE AG

(21) Appl. No.: 13/588,399

(22) Filed: Aug. 17, 2012

Related U.S. Application Data
(60) Provisional application No. 61/525,047, filed on Aug. 18, 2011.
(60) Provisional application No. 61/666,253, filed on Jun. 29, 2012.

(30) Foreign Application Priority Data
Aug. 18, 2011 (GB) 1114226.2
Jun. 29, 2012 (GB) 1211543.2

(51) Int. Cl. A61K 31/4525; A61P 1/00; C12N 5/0793; A61P 13/00; A61P 1/10; A61P 1/04; A61K 31/4709; A61P 11/00

(52) U.S. Cl. 514/304; 514/320; 435/375

(57) ABSTRACT

Described herein is a combination comprising at least one 5-HT₄ receptor agonist and at least one phosphodiesterase 4 (PDE4) inhibitor, and methods and uses thereof in the prevention and/or treatment of one or more disorders in which an increased acetylcholine release is desired; for example in the prevention and/or treatment of gastrointestinal disorders, urinary disorders, and/or respiratory disorders.

### CROSS-REFERENCE TO RELATED APPLICATIONS

The present application claims priority to U.S. Provisional Patent Application No. 61/525,047, filed Aug. 18, 2011, and U.S. Provisional Patent Application No. 61/666,253, filed Jun. 29, 2012, Great Britain Patent Application No. 1211543.2, filed on Jun. 29, 2012, and Great Britain Patent Application No. 1114226.2, filed on Aug. 18, 2011, the disclosures of which are specifically incorporated herein by reference in their entirety.

### FIELD OF THE INVENTION

The present invention relates to a composition comprising a combination of a 5-HT₄ receptor agonist and a phosphodiesterase 4 (PDE4) inhibitor, and to methods and uses thereof in the prevention and/or treatment of one or more disorders in which an increased acetylcholine release is desired; for example, in the treatment of gastrointestinal disorders, urinary disorders, and/or respiratory disorders.

### BACKGROUND TO THE INVENTION

### (1) Acetylcholine

Acetylcholine (ACh) is an important neurotransmitter of the central nervous system (CNS) as well as the peripheral nervous system (PNS) of many organisms, including humans. The PNS consists of the nerves and ganglia outside of the brain and spinal cord and is divided into the somatic nervous system, which is the system that regulates activities that are under conscious control such as body movement; and the autonomic nervous system which functions beyond our control. The autonomic nervous system is further divided into the sympathetic, parasympathetic and enteric nervous systems.

The sympathetic nervous system uses noradrenaline as the end neurotransmitter and is the system that responds to impeding danger by stimulating the cardiovascular system and inhibiting the gastrointestinal system. The parasympathetic system uses acetylcholine as the end-neurotransmitter and is responsible for the physiological response at rest, e.g. inhibition of the cardiovascular system (reduced heart rate and blood pressure) and stimulation of the gastrointestinal system.

Although the GI tract is under control of the CNS through the extrinsic nerves from the autonomic nervous system, it can function in isolation and almost all activity of the GI tract occurs involuntarily and autonomously. Its functions are being regulated by a complexly organized intrinsic nervous system, with cell bodies in the wall of the GI tract itself, the enteric nervous system (ENS). The ENS consists of two ganglionated neuronal plexuses. The plexus of Auerbach or the myenteric plexus is positioned between the longitudinal and circular muscle layer throughout the digestive tract, and continues from the oesophagus to the rectum. The plexus of Meissner or the submucosal plexus is positioned in the submucosa. The ENS integrates motility, secretion, blood flow and immune responses into organized patterns of behavior through neural reflexes in which acetylcholine plays an important role.

Acetylcholine is thus a major neurotransmitter in the autonomic/enteric nervous system, which in general activates neurons and muscles, the exact response thereof depending on the type of receptors present on the target cell. Induction of acetylcholine release may have beneficial effects on disorders where smooth muscle contraction is desired, such as gastrointestinal disorders, and disorders of the urinary system. In addition, compositions inducing acetylcholine release may be beneficial for preoperative preparation, such as for example where colonic emptying is desired.

### (2) 5-HT4 Receptors

One possible way of modulating acetylcholine release is to stimulate one or more serotonin receptors located on cholinergic nerves. Serotonin (5-hydroxytryptamine; 5-HT) is a ubiquitous signalling molecule that is involved in a variety of functions in the brain and periphery. 5-HT exerts its actions by interacting with seven receptor subtypes (5-HT₁ to 5-HT₇). All classes of the 5-HT receptor family, except for the ligand-gated 5-HT3 receptor, are members of the seven transmembrane-spanning G protein-coupled receptor family. Together with 5-HT6 and 5-HT7 receptors, 5-HT₄ receptors are positively coupled to Gₛ proteins, resulting in stimulation of adenylyl cyclase and increase in cellular cAMP. The enhanced levels of intracellular cAMP trigger a response which is cell-type specific. Such cell-type specific responses to a 5-HT4 receptor agonist include an enhanced release of neurotransmitters such as acetylcholine when the receptors are expressed on neurons, a smooth muscle relaxation when they are expressed on smooth muscle cells, and an increased contractile force for atrial cells.

It is well-established that 5-HT4 receptors are expressed on the mentioned peripheral cell types throughout the body and 5-HT4 receptor activation has been shown to be involved in many responses in different organs such as the GI tract, the heart and the urinary bladder (for review see Langlois and Fischmeister (2003)). The effect of 5-HT4 receptor activation in the GI tract has been studied extensively and the involvement of 5-HT4 receptors in peristalsis in human, rat, mouse and guinea pig is well established. Activation of 5-HT4 receptors on efferent myenteric cholinergic excitatory neurons (efferent limb of the peristaltic reflex), leading to enhanced acetylcholine release and hence increased muscle contraction, is probably the predominant mechanism by which 5-HT4 receptor agonists affect GI motility. This has been shown in many GI tissue preparations of multiple species (De Maeyer et al., 2008).

5-HT4 receptors are also expressed on human atrial and ventricular muscle cells, albeit at very low densities (Kaumann et al., 1996).

Multiple 5-HT₄ receptor agonists, such as cisapride, prucalopide, tegaserod, renzapride, mosapride and velusetrag, have/are being developed. For example prucalopride, which is the generic name for the (1:1) succinic acid addition salt of 4-amino-5-chloro-2,3-di-hydro-N-[r-(3-methoxypropyl)-4-piperidinyl]-7-benzo-furan-carboxamide, has been shown to have a strong gastrointestinal prokinetic activity.

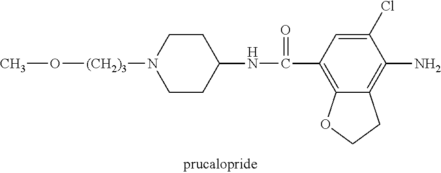

By acting on 5-HT4 receptors located on neuronal cells in the wall of the GI tract, 5-HT4 receptor agonists such as prucalopride (Resolor®) and velusetrag facilitate the release of neurotransmitters such as acetylcholine from these neurons. Additionally, for example for prucalopride there is also evidence for enhanced non-adrenergic non-cholinergic (NANC) excitatory neurotransmission. As a result of these effects, 5-HT4 receptor agonists stimulate GI motility and facilitate propulsion. For example, prucalopride is a potent and selective agonist of 5-HT4 receptors that by stimulating 5-HT4 receptors induces high amplitude propagating contractions that are propagated over the length of the colon as a peristaltic wave and therefore has significant motility enhancing effects on the large intestine. Furthermore, formulations comprising prucalopride are believed of potential use in the prevention and/or treatment of conditions associated with a poorly functioning bladder such as, e.g. urinary incontinence or urinary retention. Prucalopride is generically described in EP-0,445,862-A1, published on 11 Sep. 1991, and is specifically disclosed in WO-96/16060, published on 30 May 1996. Both the European patent application EP-0,445,862-A1, and the International patent application WO-96/16060 are herein incorporated by reference.

Although 5-HT4 receptor agonists on their own are useful for enhancing acetylcholine release, and subsequent increased muscle contraction, it would be even more beneficial if this effect could be synergistically enhanced by the addition of other pharmaceuticals that interfere with the signal transduction of presynaptic 5-HT4 receptors, making it possible to obtain similar or even increased effects with lower dosages at the location.

### (3) Phosphodiesterases (PDEs)

The pathway for a cell to degrade cAMP is via specific cyclic nucleotide phosphodiesterases (PDEs). By breaking down phosphodiester bonds, PDEs degrade second messenger molecules such as cAMP and cGMP. Therefore, inhibition of specific PDE enzymes results in a retarded break down of cAMP.

The PDE superfamily of enzymes is classified into 11 families (PDE1-PDE11), of which most are further subdivided into subfamilies. For example PDE4, 7 and 8 are predominantly cAMP hydrolases, PDE5, 6 and 9 are predominantly cGMP hydrolases, and PDE1, 2, 3, 10 and 11 can hydrolyse both cAMP and cGMP. Furthermore, due to their importance in regulating second messenger molecules, PDEs have a broad expression pattern in various tissues, cell types and subcellular locations, including expression in the heart, brain, gastrointestinal tract, blood cells, etc. However, not all PDEs are present and functional in any cell, and still little is known on the PDE subtypes involved in cAMP metabolism between different cell types. Furthermore, depending on the mechanism/receptor by which the cAMP production is triggered, different PDE subtypes can be recruited/involved in the cAMP breakdown in the given cell type. It is accordingly hard to predict which of the PDEs is involved in which pathway of which cell type.

This is also apparent from available PDE inhibitors that have been developed for various indications:

Non-selective PDE inhibitors:
Theophylline: bronchodilator
Pentoxyfylline: diabetes and peripheral nerve damage
Paraxanthine: CNS disorders

PDE1 inhibitors:
Vinpocetine: cerebrovascular disorders

PDE2 inhibitors:
EHNA: cerebrovascular disorders
Anagrelide: essential thrombocytosis

PDE3 inhibitors:
Enoximone: cardiac failure
Milrinone: cardiac failure
Levosimendan: cardiac failure

PDE4 inhibitors:
Roflumilast: COPD
Drotaverine: alleviation of renal colic pain
Rolipram: depression

In summary, acetylcholine is a major neurotransmitter in the autonomic and enteric nervous system and induction of acetylcholine release from the cholinergic neurons may have beneficial effects on disorders where smooth muscle contraction is desired. It was an object of the present invention to provide a combination capable of specifically facilitating the acetylcholine release from the cholinergic neurons while avoiding facilitation of unwanted interactions of the combination in other organs such as the cardiovascular system. In addition, the cAMP-increasing combination has to selectively target the cholinergic system, because increasing cAMP in the smooth muscle cells would result in a counteracting relaxation.

### SUMMARY OF THE INVENTION

The present invention is directed to compositions having a synergistic action between 5-HT4 receptor agonists and PDE4 inhibitors on the facilitation of acetylcholine release from cholinergic neurons towards gastrointestinal circular muscles. More importantly, this synergistic effect appears to be specific to GI cholinergic neurotransmission and the subsequent induced smooth muscle cell contraction. The compositions comprise at least one 5-HT4 receptor agonist and at least one phosphodiesterase (PDE4) inhibitor.

For example, when atrial cells are exposed to a 5-HT4 receptor agonist and a PDE4 inhibitor, no synergistic effect on atrial beating rate (chronotropy) or atrial contraction (inotropy) is observed. Atrial muscle contraction requires inhibition of PDE3 (Galindo-Tovar et al., 2009). Additionally, no unwanted GI smooth muscle relaxation occurs despite the presence of a PDE4 inhibitor. Simultaneous inhibition of PDE3 and PDE4 is necessary to induce a cAMP-mediated GI smooth muscle relaxation.

Therefore, a combination therapy of a 5-HT4 receptor agonist with a PDE4 inhibitor is a means to specifically augment the effects of a 5-HT4 receptor agonist on cholinergic neurotransmission in the GI tract, while avoiding an interaction in atrial muscle cells and avoiding unwanted PDE-induced increases in smooth muscle cAMP that would result in smooth muscle relaxation.

In an alternative embodiment, the invention is directed to a method of stimulating the release of acetylcholine from cholinergic neurons innervating gastric and/or colonic circular muscle cells. The method comprises exposing the cells for a sufficient time to a composition comprising a sufficient amount of a combination of a 5-HT₄ receptor agonist and a phosphodiesterase 4 (PDE4) inhibitor.

This invention further provides the use of a pharmaceutical composition according to this invention for the prevention and/or treatment of one or more disorders in which an increased acetylcholine release is desired such as for example selected from gastrointestinal disorders, urinary disorders, and respiratory disorders; in particular gastrointestinal disorders. Thus, in yet a further embodiment, described is a method of treating a gastrointestional disorder, urinary disorder or respiratory disorder in a patient suffering therefrom. The method comprises administering to the subject or patient an effective amount of a composition comprising a combination of a 5-HT₄ receptor agonist and a phosphodiesterase 4 (PDE4) inhibitor.

The combination therapy has thus beneficial effects on disorders in which an increased acetylcholine release is desired such as in the regulation of GI smooth muscles, including gastric circular smooth muscles, sphincters, the detrusor muscle of the urinary bladder, which are all tissues in which 5-HT4 receptor agonists have been shown to increase acetylcholine release.

In a first aspect, this invention provides a combination of a 5-HT4 receptor agonist and a phosphodiesterase 4 (PDE4) inhibitor, for use in the prevention and/or treatment of one or more disorders in which an increased acetylcholine release is desired, such as for example gastrointestinal disorders, urinary disorders, and respiratory disorders; in particular gastrointestinal disorders.

In a specific embodiment of this invention, the 5-HT4 receptor agonist is selected from the list comprising prucalopride, cisapride, dazopride, mosapride, renzapride, naronapride, zacopride, velusetrag tegaserod, metoclopramide, cinitapride, YM-53389{(+)-(S)-2-chloro-5-methoxy-4-[5-(2-piperidylmethyl)-1,2,4-oxadiazol-3-yl]aniline monohydrochloride}, RS-67333, 5-Methoxytryptamine (5-MT), and BIMU-8; in particular prucalopride.

In another specific embodiment, the phosphodiesterase 4 (PDE4) inhibitor is selected from the list comprising rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, Mesembrine, Ro20-1724, RPL-554, and YM-976; in particular roflumilast.

In a preferred embodiment, this invention provides a composition comprising the 5-HT4 receptor agonist prucalopride, and the PDE4 inhibitor roflumilast. In the context of this invention, the gastrointestinal disorder is selected from the list comprising irritable bowel syndrome, chronic constipation, constipation caused by spinal cord injury or pelvic diaphragm failure, intestinal atony, reflux esophagitis, gastroesophageal reflux disorder (GERD), Barrett syndrome, intestinal pseudoileus, acute or chronic gastritis, gastric or duodenal ulcer, Crohn's disease, non-ulcer dyspepsia, gastroparesis, functional dyspepsia, ulcerative colitis, postgastrectomy syndrome, postoperative digestive function failure, delayed gastric emptying caused by gastric neurosis, and indigestion; in particular gastroparesis, GERD, irritable bowel syndrome, constipation and intestinal atony.

In a further aspect, the present invention provides the use of a combination of a 5-HT4 receptor agonist and a PDE4 inhibitor, as defined above, in the preparation of a pharmaceutical composition for use in the prevention and/or treatment of one or more disorders in which an increased acetylcholine release is desired, such as for example selected from gastrointestinal disorders, urinary disorders, and respiratory disorders; in particular gastrointestinal disorders.

A further aspect of the present invention is to provide a pharmaceutical composition comprising a 5-HT4 receptor agonist and a phosphodiesterase 4 (PDE4) inhibitor.

In a particular embodiment, the PDE4 inhibitor is selected from the group comprising rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, Mesembrine, Ro20-1724, RPL-554, and YM-976; in particular roflumilast. In another particular embodiment, the 5-HT4 receptor agonist is selected from the group comprising prucalopride, cisapride, dazopride, mosapride, renzapride, naronapride, zacopride, velusetrag tegaserod, metoclopramide, cinitapride, YM-53389{(+)-(S)-2-chloro-5-methoxy-4-[5-(2-piperidylmethyl)-1,2,4-oxadiazol-3-yl]aniline monohydrochloride}, RS-67333, 5-Methoxytryptamine (5-MT), and BIMU-8; in particular prucalopride.

In a preferred embodiment the 5-HT4 receptor agonist is prucalopride and the PDE4 inhibitor is roflumilast.

This invention further provides the use of a pharmaceutical composition according to this invention for the prevention and/or treatment of one or more disorders in which an increased acetylcholine release is desired such as for example selected from gastrointestinal disorders, urinary disorders, and respiratory disorders; in particular gastrointestinal disorders.

In yet a further aspect, the present invention provides a method for the treatment of one or more disorders in which an increased acetylcholine release is desired, such as for example selected from gastrointestinal disorders, urinary disorders, and respiratory disorders; in particular gastrointestinal disorders; the method comprising administering to a subject in need thereof, a combination comprising a 5-HT4 receptor agonist and a phosphodiesterase 4 (PDE4) inhibitor, or a pharmaceutical composition comprising the combination.

The 5-HT4 receptor agonist and phosphodiesterase 4 (PDE4) inhibitor may be administered simultaneously, sequentially or separately to a patient in need thereof.

In yet a further aspect, the present invention provides a method of stimulating the release of acetylcholine from the cholinergic neurons innervating gastric circular muscle cells, the method comprising exposing the cholinergic neurons to a combination comprising a 5-HT4 receptor agonist and a phosphodiesterase 4 (PDE4) inhibitor. As evident from the experimental part hereinafter, when the cholinergic neuronal cells are exposed to the combination, the amount of acetylcholine released from the cells is significantly and specifically enhanced in comparison to exposure with either the 5-HT4 receptor agonist or the PDE4 inhibitor alone.

This method is in particular suitable when the release from the cholinergic neurons innervating gastric circular muscle cells, is associated with the treatment of a gastrointestinal disorder.

This invention also provides a method of treating a lack of gastric motility comprising administering to a patient in need thereof a sufficient amount of a 5-HT4 receptor agonist and a PDE4 inhibitor; wherein the 5-HT4 receptor agonist and the PDE4 inhibitor may be administered simultaneous, sequential or separate to a patient in need thereof. In an even further embodiment, the invention also provides a method of treating a lack of gastric motility comprising administering to a patient in need thereof a sufficient amount of a composition comprising a 5-HT4 receptor agonist and a PDE4 inhibitor.

Both the foregoing general description and the following brief description of the drawings and detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIGS. 1 (A) and (B): Shows the influence of prucalopride (Pru; 0.01 μM, A; 0.03 μM, B), IBMX and prucalopride in the presence of IBMX on the S2/S1 ratio of electrical field stimulation (EFS)-evoked total radioactivity release from gastric tissue. Tissues were stimulated twice (S1 and S2; 15V, 1 ms, 4 Hz, 2 min); IBMX was added 36 min and prucalopride 15 min before S2. The EFS-induced efflux of total radioactivity above baseline by S2 is expressed as a ratio of that by S1. Means±SEM of n=5 to 6 tissues are shown. *P<0.05: significantly different from control; #P<0.05, ###P<0.001: significantly different from prucalopride alone.

FIG. 1(C): Shows the influence of 0.01 μM prucalopride (Pru), 0.3 μM roflumilast (Roflu) and prucalopride in the presence of roflumilast on the S2/S1 ratio of EFS-induced total radioactivity release from gastric tissue. Tissues were stimulated twice (S1 and S2; 15 V, 1 ms, 4 Hz, 2 min). Roflumilast was added 36 min and prucalopride was added 15 min before S2. Means±SEM of the S2/S1 ratio of n=6 tissues are shown. ***p<0.001; *p<0.05: significantly different from control (0.1% DMSO). ###p<0.001: significantly different from 0.01 μM prucalopride. ^{ooo} P<0.001: significantly different from 0.3 μM roflumilast (ANOVA followed by a Bonferroni multiple comparisons t-test; 5 comparisons ie DMSO-Pru, Roflu and Roflu-Pru versus DMSO, Roflu-Pru versus DMSO-Pru and Roflu-Pru versus Roflu).

FIG. 1(D): Shows the influence of 0.01 μM velusetrag (Velu), 1 μM rolipram (Roli) and velusetrag in the presence of rolipram on the S2/S1 ratio of EFS-induced outflow of total radioactivity from gastric tissue. Tissues were stimulated twice (S1 and S2; 15 V, 1 ms, 4 Hz, 2 min). Rolipram was added 36 min and velusetrag was added 15 min before S2. Means±SEM of the S2/S1 ratio of n=6−7 tissues are shown. ***p<0.001; **p<0.01: significantly different from control (0.01% DMSO-0.1% DMSO). ###p<0.001: significantly different from rolipram 1 μM, ^{ooo} P<0.001: significantly different from velusetrag 0.01 μM. (ANOVA followed by a Bonferroni multiple comparisons t-test; 5 comparisons ie DMSO-Velu, Roli-DMSO and Roli-Velu versus DMSO, Roli-Velu versus DMSO-Velu and Roli-Velu versus Roli-DMSO).

FIG. 2: Shows the influence of prucalopride (Pru, 0.01 μM), rolipram (Roli, 1 μM) and prucalopride in the presence of rolipram on the S2/S1 ratio of EFS-evoked total radioactivity release from gastric tissue. Tissues were stimulated twice (S1 and S2; 15V, 1 ms, 4 Hz, 2 min); rolipram was added 36 min and prucalopride 15 min before S2. The EFS-induced efflux of total radioactivity above baseline by S2 is expressed as a ratio of that by S1. Means±SEM of n=6 tissues are shown. ###P<0.001: significantly different from prucalopride alone.

FIG. 3: Shows the representative trace (auxotonic registration) demonstrating the facilitating effect of 0.1 μM prucalopride on submaximal EFS-induced contractions in the presence of 300 μM L-NAME in gastric muscle strips.

FIG. 4: Shows the enhancing effect of increasing concentrations of prucalopride (Pru) on EFS-induced submaximal contractions in gastric muscle strips. Responses are expressed as percentage of the mean of the 5 contractions before adding prucalopride. Means±SEM of n=6 tissues are shown. ***P<0.001, *P<0.05: significant difference of the final response versus that in control tissues without prucalopride.

FIG. 5: Shows the influence of increasing concentrations of the PDE-inhibitors IBMX (B), cilostamide (C), and rolipram (D) on EFS-induced submaximal contractions in gastric muscle strips. Six trains of EFS were applied in the presence of each concentration of PDE-inhibitor and the response to the 6^{th} train was expressed as percentage of the mean of the 5 contractions before adding the lowest concentration of the PDE-inhibitor. Control tissues (A) were stimulated 47 times and the response was measured at each 6^{th} train from train 11 (T11) on. Means±SEM of n=6-8 tissues are shown. ***P<0.001, **P<0.01, *P<0.05: significant difference versus the response before.

FIG. 6: Shows the representative trace (isometric registration) demonstrating the influence on submaximal EFS-induced contractions of consecutive administration of 1 μM rolipram and 1 μM cilostamide (A) in gastric muscle strips.

FIG. 7: Shows the influence of IBMX (1 or 3 μM) on the enhancing effect of 0.01 μM prucalopride (Pru) on EFS-induced submaximal contractions in gastric muscle strips. Responses are expressed as % of the mean of the 5 contractions before adding prucalopride. Means±SEM of n=6 tissues are shown. ***P<0.001: significant difference of the final response versus that in control tissues without prucalopride; #P<0.05: significant difference of the final response versus that in tissues only treated with prucalopride.

FIG. 8: Shows the influence of 1 μM rolipram on the enhancing effect of 0.01 (A), 0.03 (B) and 0.1 (C) μM prucalopride on EFS-induced submaximal contractions in gastric muscle strips. Responses are expressed as percentage of the mean of the 5 contractions before adding rolipram. Means±SEM of n=7-8 tissues are shown. ***P<0.001, *P<0.05: significant difference of the final response versus that in control tissues without prucalopride.

FIG. 9: Shows the influence of increasing concentrations of the PDE inhibitors IBMX (B), vinpocetine (C), EHNA (D), cilostamide (E) and zaprinast (F) on EFS (10 s trains at 4 Hz; 0.25 ms; V50%) induced submaximal contractions in colon circular muscle tissue. Six trains of EFS were applied in the presence of each concentration of PDE-inhibitor and the response of the 6^{th} train was expressed as percentage of the mean of the 5 contractions before adding the lowest concentration of the PDE inhibitor. Control tissues (A) were stimulated 41 times and the response was measured at each 6^{th} train from train 11 (T11) on. Means±S.E.M. of n=6-7. *P<0.05; **P<0.01; ***P<0.001: significant difference versus before (repeated measures ANOVA followed by a Bonferroni corrected t-test)

FIG. 10: Shows the influence of increasing concentrations of the PDE4 inhibitor rolipram (B) on EFS (10 trains at 4 Hz; 0.25 ms; V50%) induced submaximal contractions in colon circular muscle tissue, expressed as described in the legend of FIG. 1. Parallel time controls, not receiving an agent (A), tissues receiving the 50% ethanol dilution series as for IBMX (C) and tissues receiving the DMSO dilution series as for rolipram, cilostamide and vinpocetine (D) are also shown. Means±S.E.M. of n=4-6. *P<0.05; **P<0.01; ***P<0.001: significant difference versus before (repeated measures ANOVA followed by a Bonferroni corrected t-test)

FIG. 11: Shows the facilitating effect of 1 μM prucalopride (PRU) on EFS-induced submaximal cholinergic contractions in colon circular muscle tissue in the presence of PDE inhibitors IBMX 0.3 μM (A) or 1 μM (B), or rolipram 3 μM (C). Means±S.E.M. of n=5-8. *P<0.05; **P<0.01; ***P<0.001: significant difference of the response at stimulation train 13 (2^{nd} stimulation train after adding prucalopride) versus that in control tissues without prucalopride (one-way ANOVA followed by a Bonferroni corrected t-test)

FIG. 12 (A): Shows the representative trace of a colon circular muscle tissue showing the influence on submaximal EFS-induced contractions of consecutive administration of 1 μM prucalopride and 3 μM rolipram. (B) Mean (±S.E.M.; n=8) result of the experiment shown in panel A, and in parallel tissues only receiving prucalopride, or no substance at all (time control).**P<0.01: significant difference of the response to stimulation train 7 (2^{nd} stimulation train after adding prucalopride) versus the mean response to stimulation train 3-5 just before adding prucalopride (paired t-test). ∇P<0.01: significant difference of the response to stimulation train 19 (2^{nd} stimulation train after adding rolipram) versus the mean response to stimulation train 15-17 (paired t-test)

### DETAILED DESCRIPTION OF THE INVENTION

In an exemplary embodiment, the present invention is directed to a combination of at least one 5-HT₄ receptor agonist and at least one phosphodiesterase 4 (PDE4) inhibitor, and a pharmaceutical composition comprising such a combination. The combination is useful, for example, in the prevention and/or treatment of one or more disorders in which an increased acetylcholine release is desired.

Reference to a 5-HT₄ receptor agonist and/or a PDE4 inhibitor shall at all times be understood to include all active forms of such agents, including the free form thereof (e.g., free and/or base form) and also all pharmaceutically acceptable salts, polymorphs, hydrates, silicates, stereo-isomers and so forth. Active metabolites, in any form, are also meant to be included.

The present invention is described herein using several definitions, as set forth below and throughout the application.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “5-HT₄ receptor agonist” as used herein, is meant to include any agent that has an affinity for serotonin type-4 receptors and is able to mimic the stimulating effects of serotonin at this specific cellular receptor, as e.g. is useful in the prevention and/or treatment of certain gastrointestinal diseases. Examples of 5-HT₄ receptor agonists include but are not limited to prucalopride, cisapride, dazopride, mosapride, renzapride, naronapride, zacopride, velusetrag tegaserod, metoclopramide, cinitapride, YM-53389{(+)-(S)-2-chloro-5-methoxy-4-[5-(2-piperidylmethyl)-1,2,4-oxadiazol-3-yl]aniline monohydrochloride}, RS-67333 (http://en.wikipedia.org/wiki/RS-67,353), 5-Methoxytryptamine (5-MT), and BIMU-8 (http://en.wikipedia.org/wiki/BIMU8).

The term “phosphodiesterase 4 (PDE4) inhibitor” as used herein, is meant to include any agent which inhibits the activity of PDE4 in a selective manner, i.e. which does not substantially modulate the activity of any of the other PDE family members. In particular, inhibition of PDE4 results in blocking the hydrolysis of cAMP, thereby increasing levels of cAMP within cells. Examples of PDE4 inhibitors include, but are not limited to, rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, Mesembrine, Ro20-1724, RPL-554, and YM-976. See http://en.wikipedia.org/wiki/PDE4 inhibitor.

Reference to a 5-HT4 receptor agonist and/or a PDE4 inhibitor shall at all times be understood to include all active forms of such agents, including the free form thereof (e.g. free and/or base form) and also all pharmaceutically acceptable salts, polymorphs, hydrates, silicates, stereo-isomers and so forth. Active metabolites, in a form, are also meant to be included.

The phrase “disorder in which an increased acetylcholine release is desired” is meant to include any disorder which may be treated and/or prevented by increasing the acetylcholine release above basal. Such disorders may include, but are not limited to, gastrointestinal disorders, urinary disorders, and respiratory disorders.

Compositions of the Invention

In an exemplary embodiment, the present invention is directed to a novel combination which synergistically increases acetylcholine release from cholinergic nerve endings in the peripheral nervous system, thereby stimulating GI (e.g. gastric or colonic) smooth muscle contraction while avoiding undesired GI smooth muscle relaxation through increased cAMP levels and undesired contraction/relaxation in cardiac muscles. The combination, or a composition comprising such a combination, comprises at least one 5-HT₄ receptor agonist and at least one phosphodiesterase 4 (PDE4) inhibitor. Administration of both of these therapeutic agents results in a potentiation of the effect of the 5-HT₄ receptor agonist; administration of both agents therefore produces an effect that is larger than that of the 5-HT₄ receptor agonist alone or the PDE4 inhibitor alone.

Currently available 5-HT₄ receptor agonist pharmaceutical compositions include prucalopride, which is available in a once-daily tablet form containing 2 or 1 mg of prucalopride. Currently available PDE4 inhibitor pharmaceutical compositions include roflumilast, which is available in a once-daily tablet form containing 500 μg roflumilast. According to one embodiment of the invention, the composition comprises separate, individual dosage forms of the 5-HT₄ receptor agonist and PDE4 inhibitor. Alternatively, the composition can comprise a combination of those therapeutic agents in a singular dosage form.

The present invention provides for administering each of the aforementioned therapeutics, i.e. the 5-H T4 receptor agonist and the PDE4 inhibitor, as part or the same therapeutic treatment program or regimen. Accordingly, the present invention also provides compositions comprising a 5-HT4 receptor agonist and a PDE4 inhibitor.

In an exemplary embodiment, the 5-HT₄ receptor agonist is prucalopride, and the PDE4 inhibitor is roflumilast. This combination may be used for the prevention and/or treatment of gastrointestinal disorders.

The compositions of the invention can be formulated into any pharmaceutically acceptable dosage form, such as oral tablets, liquid dispersions, gels, aerosols, ointments, creams, capsules, sachets, solutions, dispersions and mixtures thereof. In addition, the composition can be formulated into a controlled release formulation, fast melt formulation, lyophilized formulation, delayed release formulation, extended release formulation, pulsatile release formulation, mixed immediate release and controlled release formulation, etc.

The compositions of the invention can additionally comprise one or more pharmaceutically acceptable excipients, carriers, or a combination thereof.

Suitable dosages of 5-HT₄ receptor agonists and PDE4 inhibitors are known in the art. In addition, dosing of the compositions of the invention can be one or more times daily, including 2, 3, 4, or 5× or more daily. Dosing can also be for any desired time period, such as 1, 2, 3, 4, 5, 6, or 7 days; 1, 2, 3, 4, or 5 weeks, 1, 2, 3, 4, 5, 6, 7, 8, 9, 10, 11, or 12 months, or any combination thereof. Dosing can also continue over a year or more period.

5-HT₄ receptor agonists can be used in the compositions of the invention at any pharmaceutically acceptable dosage, including but not limited to, daily or individual dosages of about 50, about 100, about 200, about 300, about 400, about 500, about 600, about 700, about 800, about 900, or about 1000 mcg; or about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 5, about 6, about 7, about 8, about 9, about 10 mg, about 15, about 16, about 17, about 18, about 19, about 20 mg, about 21, about 22, about 23, about 24, about 25, about 26, about 27, about 28, about 29, about 30 mg, about 35, about 40, about 45, about 50, about 55, about 60, about 65, about 70, about 75, about 80, about 85, about 90, about 95, about 100 mg, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, or about 250 mg.

For example, the recommended dosage of procalopride in adults is 2 mg administered orally once daily; exceeding this dosage is not expected to increase efficacy. The recommended starting dose in elderly patients (>65 years) is 1 mg once daily; thereafter the dosage can be increased to 2 mg once daily, if needed. See http://en.widipedia.org/widi/Prucalopride. Accordingly, exemplary dosages of prucalopride in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 mg.

Dosages of the 5-HT₄ receptor agonist cisapride range from 10-20 mg orally 4 times a day 15 minutes before meals and at bedtime for Gastroesophageal Reflux Disease and Gastroparesis, 5-10 mg orally 3 times a day 15 minutes before meals for Dyspepsia, with the dosage reduced by 50% for subjects with liver complications. For children older than 1 year, dosages are 0.2 to 0.3 mg/kg/dose orally 3 to 4 times a day, with a maximum of 10 mg/dose (e.g. for Gastroesophageal Reflux Disease). See http://www.drugs.com/dosage/cisapride.html. Accordingly, exemplary dosages of cisapride in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 35, about 40, about 45, about 50, about 55, about 60, about 65, about 70, about 75, about 80, about 85, or about 90 mg.

Dosages of the 5-HT₄ receptor agonist mosapride are generally 5 mg 3 times/day. See http://www.mims.com/USA/Drug/Info/mosapride

Accordingly, exemplary dosages of mosapride in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg.

Dosages of the 5-HT₄ receptor agonist renzapride of 4 mg/day group have been shown to show consistently numerically greater results than placebo in a clinical trial for constipation-predominant irritable bowel syndrome. See http://www.ncbi.nlm.nih.gov/pubmed/18284648. Accordingly, exemplary dosages of mosapride in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, or about 20 mg.

Dosages of the 5-HT₄ receptor agonist naronapride used in a recent Phase 2 clinical trial were 80 mg twice daily in healthy adult males. See http://www.aryx.com/wt/page/ati7505. Accordingly, exemplary dosages of naronapride in the compositions of the invention, to be administered one or more times daily, include, but are not limited to, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, or about 250 mg.

Dosages of the 5-HT₄ receptor agonist velusetrag described in a clinical trial included 15-50 mg daily. See http://www.ncbi nlm.nih.gov/pubmed/19691492. Accordingly, exemplary dosages of velusetrag in the compositions of the invention, to be administered one or more times daily, include, but are not limited to, about 1, about 2, about 3, about 4, about 5, about 6, about 7, about 8, about 9, about 10, about 20, about 30, about 40, about 50, about 60, about 70, about 80, about 90, about 100, about 110, about 120, about 130, about 140, or about 150 mg.

Dosages of the 5-HT₄ receptor agonist tegaserod is generally 6 mg twice daily for four to six weeks. See http://digestive-system.emedtv.com/tegaserod/tegaserod-dosing.html. Accordingly, exemplary dosages of tegaserod in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20 mg, about 21, about 22, about 23, about 24, about 25, about 26, about 27, about 28, about 29, or about 30 mg.

Dosages of the 5-HT₄ receptor agonist metoclopramide range from 10 to 15 mg up to 4 times a day (oral, adult dose for Gastroesophageal Reflux Disease), and 0.4 to 0.8 mg/kg/day in 4 divided doses (oral, IM, IV, infants and children for Gastroesophageal Reflux Disease). See http://www.drugs.com/dosage/metoclopramide.html. Accordingly, exemplary dosages of metoclopramide in the compositions of the invention, to be administered one or more times daily, include, but are not limited to, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, about 15, about 16, about 17, about 18, about 19, about 20 mg, about 21, about 22, about 23, about 24, about 25, about 26, about 27, about 28, about 29, about 30 mg, about 35, about 40, about 45, about 50, about 55, about 60, about 65, about 70, about 75, about 80, about 85, about 90, about 95, or about 100 mg.

Dosages of the 5-HT₄ receptor agonist cinitapride are generally 1 mg orally 3 times a day for adults. See http://www.medindia.net/doctors/drug information/cinitapride.htm. Accordingly, exemplary dosages of cinitapride in the compositions of the invention, to be administered one or more times daily, include, but are not limited to, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10 mg.

PDE4 inhibitors can be used in the compositions of the invention at any pharmaceutically acceptable dosage, including but not limited to, daily or individual dosages of about 50, about 100, about 200, about 300, about 400, about 500, about 600, about 700, about 800, about 900, or about 1000 mcg; or about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0, about 5, about 6, about 7, about 8, about 9, about 10 mg, about 15, about 16, about 17, about 18, about 19, about 20 mg, about 21, about 22, about 23, about 24, about 25, about 26, about 27, about 28, about 29, about 30 mg, about 35, about 40, about 45, about 50, about 55, about 60, about 65, about 70, about 75, about 80, about 85, about 90, about 95, about 100 mg, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, or about 250 mg.

Roflumilast, a PDE4 inhibitor, is currently approved for treating COPD, and the approved dosage is one 500-mcg (microgram) daily dose. See http://lungs.emedtv.com/roflumilast/roflumilast-dosage.html. Accordingly, exemplary dosages of roflumilast in the compositions of the invention, to be administered one or more times daily, include, but are not limited to, about 100, about 200, about 300, about 400, about 500, about 600, about 700, about 800, about 900, or about 1000 mcg.

Dosages of drotaverine, a PDE4 inhibitor, are typically 40-80 mg, twice daily (adults), 20 mg, 3-4 times daily (children 1-6 years), and 40 mg twice daily (children greater than 6 years). See http://www.mims.com/USA/drug/info/drotaverine/?q=other%20d rugs%20acting%20on%20the%20genito-urinary%20system&type-full. Accordingly, exemplary dosages of drotaverine in the compositions of the invention, to be administered one or more times daily, include, but are not limited to, about 5, about 6, about 7, about 8, about 9, about 10 mg, about 11, about 12, about 13, about 14, about 15, about 16, about 17, about 18, about 19, about 20 mg, about 21, about 22, about 23, about 24, about 25, about 26, about 27, about 28, about 29, about 30 mg, about 35, about 40, about 45, about 50, about 55, about 60, about 65, about 70, about 75, about 80, about 85, about 90, about 95, about 100 mg, about 110, about 120, about 130, about 140, about 150 mg, about 160, about 170, about 180, about 190, about 200, about 210, about 220, about 230, about 240, or about 250 mg.

Disorders to be Prevented and/or Treated

An exemplary embodiment of the present invention is the combination of the 5-HT4 receptor agonist, prucalopride, and the PDE4 inhibitor roflumilast. For example, the combination of the 5-HT4 receptor agonist, prucalopride, and the PDE4 inhibitor roflumilast may be used for the prevention and/or treatment of gastrointestinal disorders associated to an increase of acetylcholine release.

This invention provides the use of a combination of a 5-HT4 receptor agonist and a PDE4 inhibitor for the prevention and/or treatment of one or more disorders in which an increased acetylcholine release is desired, such as for example, gastrointestinal disorders, urinary disorders, and respiratory disorders. In particular the use of a 5-HT4 receptor agonist and a selective PDE4 inhibitor for the prevention and/or treatment of one or more disorders in which an increased acetylcholine release in the peripheral nervous system is desired.

In an exemplary embodiment, this invention provides the use of a combination of a 5-HT4 receptor agonist and a PDE4 inhibitor for the prevention and/or treatment of gastrointestinal disorders in which an increased acetylcholine release is desired.

Exemplary disorders treated or prevented by an increased acetylcholine release, include, but are not limited to, gastrointestinal disorders, urinary disorders, and respiratory disorders.

Gastrointestinal disorders in which an increased acetylcholine release might be desired, include, but are not limited to, irritable bowel syndrome, chronic constipation, constipation caused by spinal cord injury or pelvic diaphragm failure, intestinal atony, reflux esophagitis, gastroesophageal reflux disorder (GERD), Barrett syndrome, intestinal pseudoileus, acute or chronic gastritis, gastric or duodenal ulcer, Crohn's disease, non-ulcer dyspepsia, gastroparesis, functional dyspepsia, ulcerative colitis, postgastrectomy syndrome, postoperative digestive function failure, delayed gastric emptying caused by gastric neurosis, and indigestion; in particular gastroparesis, GERD, irritable bowel syndrome, constipation and intestinal atony.

The current invention also provides a method for the prevention and/or treatment of one or more disorders in which an increased acetylcholine release is desired; the method comprising administering to a subject in need thereof, a combination of a 5-HT4 receptor agonist and a PDE4 inhibitor. The 5-HT4 receptor agonist and PDE4 inhibitor may be administered simultaneously, sequentially or separately to a patient in need thereof. An exemplary method according to the present invention comprises administering each of the aforementioned therapeutics, i.e., the at least one 5-HT₄ receptor agonist and the at least one PDE4 inhibitor, as part of the same therapeutic treatment program or regimen. The 5-HT₄ receptor agonist and PDE4 inhibitor may be administered simultaneously or sequentially (starting with either the 5-HT₄ receptor agonist or the PDE4 inhibitor).

Another exemplary method of the invention provides a combination according to this invention, a composition according to this invention, or a method for stimulating the release of acetylcholine from cholinergic neurons innervating gastric and/or colonic smooth muscle cells, the method comprising 5-HT₄ receptor agonist and PDE4 inhibitor, wherein when the cholinergic neurons are exposed to the combination or compositi0on, the amount of acetylcholine released from the cholinergic neurons is greater than when the cholinergic neurons are individually exposed to either the 5-HT₄ receptor agonist or the PDE4 inhibitor alone, under the same conditions and for the same time.

The amount of acetylcholine released upon exposure to the therapeutic agents of the present invention is equal to or greater than about 5, about 10, about 15, about 20, about 25, about 30, about 35, about 40, about 45, about 50, about 60, about 70, about 80, about 90, about 100, about 125, about 150, about 175, about 200, about 250, about 300, about 500, about 750, and about 1000 percent of the amount of acetylcholine released after neuronal cells are exposed to only the same 5-HT₄ receptor agonist or only the same PDE4 inhibitor alone, under the same conditions and for the same time.

An additional exemplary embodiment is a method for treating a lack of gastric and/or colonic motility comprising administering to a patient in need thereof a sufficient amount of a composition according to the invention. In particular, the present invention encompasses a method of treating a lack of gastric motility comprising administering to a patient in need thereof a sufficient amount of a composition according to this invention.

In yet a further embodiment, the present invention provides a method of selectively stimulating gastric and/or colonic smooth muscle cell contraction, the method comprising exposing cholinergic neurons innervating the smooth muscle cell with an effective amount of a combination or a composition according to this invention, and releasing acetylcholine from the cholinergic neurons towards the cell to stimulate contraction, wherein substantially no cAMP-mediated smooth muscle relaxation and/or atrial muscle contraction occurs.

Other exemplary methods for using the combination or a composition according to this invention include methods for pre-operative preparation of patients, where, for example, colonic emptying is desired prior to diagnostic or surgical procedures; methods for preventing patients from straining at defecation; methods for maintaining, both before and after surgery, soft feces in patients with hemorrhoids and other anorectal disorders; and methods for treating drug overdose and poisoning by stimulating the removal of unwanted agents from the intestine.

Kits Comprising Compositions of the Invention

Accordingly in a further aspect the present invention provides a method of selectively stimulating gastric and/or colonic smooth muscle cell contraction, the method comprising exposing cholinergic neurons innervating the smooth muscle cell with an effective amount of a combination or a composition as described herein, and releasing acetylcholine from the cholinergic neurons towards the cell to stimulate contraction, wherein substantially no cAMP-mediated smooth muscle relaxation and/or atrial muscle contraction occurs.

The combination according to the invention may be formulated into a kit. The kit may comprise a container for containing the separate compositions such as a divided bottle or a divided foil packet, wherein each compartment contains a plurality of dosage forms (e.g. tablets) comprising either the at least one 5-HT₄ receptor agonist or the at least one PDE4 inhibitor. Alternatively, rather than separating the active ingredient-containing dosage forms, the kit may contain separate compartments each of which contains whole dosage which comprises separate compositions. An example of this type of kit is a blister pack wherein each individual blister contains two tablets, one tablet comprising the 5-HT₄ receptor agonist, the other comprising the PDE4 inhibitor. Typically the kit comprises directions for the administration of the separate components. Such instructions would cover situations such as: (i) the dosage form in which the components are administered (e.g. oral and parenteral), (ii) when the component parts of the product are administered at different dosage intervals, or (iii) when titration of the individual components of the combination is desired by the prescribing physician. The container having deposited thereon a label that describes the contents therein and any appropriate warnings.

According to yet another method of treating patients with the combination of this invention, the combination, or composition comprising the combination is packaged with a memory aid on the kit, e.g., in the form of numbers next to the tablets or capsules whereby the numbers correspond with the days of the regimen during which the tablets or capsules so specified should be ingested. Another example of such a memory aid is a calendar printed on the card e.g. as follows “First Week, Monday, Tuesday, Wednesday, Thursday, Friday, Saturday, and Sunday; Second Week, Monday, Tuesday, Wednesday, Thursday, Friday, Saturday, and Sunday” etc. Other variations of memory aids will be readily apparent.

A “daily dose” can be a single tablet or capsule or several pills or capsules to be taken on a given day. Also a daily dose of the first compound can consist of one tablet or capsule while a daily dose of the second compound can consist of several tablets or capsules and vice versa. The memory aid should reflect this.

This invention will be better understood by reference to the Examples that follow, but those skilled in the art will readily appreciate that these are only illustrative of the invention as described more fully in the claims that follow thereafter. Additionally, throughout this application, various publications are cited. The disclosure of these publications, including but not limited to patents and published patent applications, is hereby incorporated by reference into this application to describe more fully the state of the art to which this invention pertains.

### EXAMPLES

### Part A

### Gastric Circular Muscles Experiments

### Example 1

### Preparation of Test Animals

For experiments in examples 5 and 8 (experiments without PDE-inhibitors), stomachs were obtained from approximately 6 months old healthy castrated male pigs, slaughtered at a local abattoir; the stomachs were transported to the laboratory in ice-chilled physiological salt solution. For experiments in examples 6, 7, 9 and 10 (experiments with PDE-inhibitors), approximately 2 month old male piglets (Line 36, weighing approximately 20 kg) were obtained from Rattlerow Seghers (Lokeren, Belgium). On the morning of the experiment, these 2 month old piglets were anesthetized with an intramuscular injection of 5 ml Zoletil 100 (containing 250 mg tiletamine and 250 mg zolazepam). After exsanguination, the entire stomach was dissected.

For preparation of the smooth muscle strips, the stomach was cut open along the lesser curvature and placed in physiological salt solution (PSS) at room temperature (composition in mM: 112 NaCl, 4.7 KCl, 1.2 MgCl₂, 1.2 KH₂PO₄, 2.5 CaCl₂, 11.5 glucose and 25 NaHCO₃ as described by Mandrek and Milenov [1991; PSS I]; or 118 NaCl, 4.69 KCl, 1.18 MgSO₄, 1.18 KH₂PO₄, 2.51 CaCl₂, 11.1 glucose, 25 NaHCO₃ [Krebs-Henseleit; PSS II). After removal of the mucosa:
4 to maximum 12 muscle strips of approximately 1.5 cm in length and 0.3 cm in width were prepared from the proximal stomach in the direction of the circular muscle layer;
up to 6 strips were obtained from the ventral side cutting from the great curvature towards the small one;
the additional strips were prepared at the same level cutting in the direction of the circular muscle layer over the great curvature so that these strips were partially from the ventral and partially from the dorsal side.

Strips used for release experiments were always obtained from the ventral side. All strips were used on the day of preparation. For functional experiments with measurement of contractility, the strips were mounted under a load of 2 g between 2 platinum plate electrodes in classic organ baths containing:
10 ml of PSS I (experiments without PDE-inhibitors),
5 ml of PSS II (experiments with PDE-inhibitors other than IBMX)
7 ml of PSS II (experiments with IBMX)
   at 37° C. and gassed with carbogen (95% O₂/5% CO₂). Mechanical activity was recorded auxotonically via a Grass force-displacement transducer FT03 coupled in series with a 1 g cm⁻¹ spring on a Graphtec linearcorder F WR3701 in the first part of the study; in the second part of the study, mechanical activity was recorded isometrically via a Grass force-displacement transducer FT03 (experiments with IBMX) or a MLT050/D force transducer from ADInstruments (experiments with other PDE-inhibitors) on a PowerLab/8 sp data recording system (ADInstruments) with Chart software.

For release experiments, strips were mounted between 2 platinum wire electrodes under a load of 2 g in 2 ml organ baths containing PSS I, to which also 0.0015 mM choline and 0.057 mM ascorbic acid was added. Electrical field stimulation was performed by means of a Grass S88 stimulator with a constant voltage unit or a 4 channel custom-made stimulator

### Example 2

### Methodology for Studying EFS-Induced Contraction of Gastric Muscles

In all series without PDE-inhibitors where electrically induced contractions were studied (example 8), the PSS I continuously contained 4 μM guanethidine and 300 μM N^{G}-nitro-L-arginine methyl ester (L-NAME) to avoid noradrenergic and nitrergic responses respectively; additionally it contained 10 μM indomethacine to avoid spontaneous progressive contraction due to release of prostaglandins. After at least 1 h of equilibration with rinsing every 15 min, the tissues were contracted with 3 μM carbachol to test the contractile reactivity of the strip; this was followed by rinsing every 10 min during 30 min. Electrical field stimulation (EFS) was then applied twice at an interval of 5 min (10 s train at supramaximal voltage, 0.5 ms and 4 Hz). This yielded reproducible contractions after which 10s trains of EFS were applied at 5 min interval with decreasing voltage until the voltage yielding a contraction amplitude of approximately 50% of that obtained at supramaximal voltage (V50% C) was reached. EFS was then stopped for 30 min with rinsing every 10 min. EFS was then started again and 10 s trains at V50% C, 0.5 ms and 4 Hz were repeated at 5 min interval until stabilization. After a further 5 trains, 0.03, 0.1 or 0.3 μM prucalopride was added to 3 parallel tissues and 10 further trains were registered; a fourth tissue received the solvent of prucalopride (control). To test antagonists versus the effect of prucalopride, the antagonist was added after 5 trains at V50% C; 6 further trains were then obtained before adding 0.3 μM prucalopride and registering 10 further trains; a parallel control strip received the solvent of the antagonist. To evaluate the neurogenic and cholinergic nature of the EFS-induced contractions, the influence of 3 μM tetrodotoxin and 1 μM atropine was tested respectively. To test the possible influence of prucalopride on contractions induced by exogenous acetylcholine, a cumulative concentration-response curve to acetylcholine was constructed with half log unit ascending concentration increments from 1 nM onwards; after rinsing for 1 h at 10 min intervals, 0.03, 0.1 or 0.3 μM prucalopride was incubated for 15 min and the concentration-response curve to acetylcholine was repeated.

In experiments with PDE-inhibitors (examples 9 and 10), the PSS II continuously contained 100 μM N^{G}-nitro-L-arginine methyl ester (L-NAME) and 1 μM indomethacine. The initial part of the protocol with carbachol and EFS to determine the V50% C was as described above except that trains of EFS were administered every 3 min. Once EFS was started again at V50% C (0.5 ms, 4 Hz, 10 s) and 5 stable responses were obtained, 2 types of experiments were performed.
1. The influence of the PDE-inhibitors IBMX, vinpocetine, EHNA, cilostamide and rolipram on the half maximal electrically induced contractions was investigated by adding them in half log unit ascending concentrations, starting after the 5^{th} train and registering the response to 6 trains after addition of each concentration. The influence of cilostamide plus rolipram was tested by adding 1 μM cilostamide, registering 10 trains, then adding 1 μM rolipram and registering another 20 trains; in half of the tissues the order of administration was reversed.
2. The influence of IBMX and rolipram versus prucalopride was studied as follows. A total of 33 to 35 trains (10s, V50% C, 0.5 ms, 4 Hz) was delivered at 3 min intervals. After 5 trains, 1, 3 or 10 μM IBMX was administered and after 15 trains 0.01 μM prucalopride; control tissues only received prucalopride or solvent. Similarly, 1 μM rolipram was added after 5 trains and 0.01, 0.03 or 0.1 μM prucalopride was added after 15 trains; in a small number of tissues, rolipram was added after 20 trains in the presence of prucalopride had been obtained.

### Example 3

### Methodology for Analyzing EFS-Induced Acetylcholine Release from Cholinergic Neurons Innervating Pig Gastric Muscle

The same method was used as described before (Leclere and Lefebvre, 2001). Strips were equilibrated for 1 h with superfusion of PSS I at 2 ml min⁻¹ (Gilson Minipuls, France) and continuous EFS (40 V, 1 ms, 0.5 Hz) was applied for the last 20 min. Superfusion was stopped and the strips were incubated for 30 min with [³H]-choline (5 μCi ml⁻¹) under continuous EFS (40 V, 1 ms, 2 Hz). EFS was stopped and the tissues were then superfused (2 ml min⁻¹) for 90 min to remove loosely bound radioactivity with PSS I, from now on also containing 10 μM hemicholinium-3 to prevent re-uptake of choline, 10 μM physostigmine to prevent hydrolysis of acetylcholine and 1 μM atropine to prevent auto-inhibition of acetylcholine release. After washout, the organ bath was filled with 1 ml of PSS. This was collected and replaced at 3 min intervals for a total of 37 samples. The strips were stimulated twice (S1 and S2) at 15 V, 1 ms and 4 Hz for 2 min starting at the 13^{th} (sample 5) and 73^{rd} (sample 25) min after the end of the washout period. Prucalopride (0.03, 0.1 or 0.3 μM) was added 15 min (sample 20) before S2. The 5-HT₄ receptor antagonist GR113808 (1, 10 or 100 nM) was tested versus 0.3 μM prucalopride by adding it 21 min (sample 13) before prucalopride. In the second part of the study, the influence of 10 μM IBMX, added from sample 13 onwards, was tested versus 0.01 or 0.03 μM prucalopride, added from sample 20 onwards. In the same protocol, the influence of 10 μM vinpocetine, 10 μM EHNA, 1 μM cilostamide and 1 μM rolipram was tested versus 0.01 μM prucalopride. At the end of the experiment, the tissues were blotted and weighed. For each sample, 0.5 ml was mixed with 2 ml of the scintillator containing solution Ultima Gold (Perkin Elmer, USA). Radioactivity of all samples was measured by liquid scintillation counting (Packard Tri-Carb 2100 TR, Packard Instrument Company, USA); external standardization was used to correct for counting efficiency.

### Example 4

### Data Collection

This example summarizes how the data collected in Examples 1-3 was analyzed. In the contractility study, the average contraction to 5 trains of EFS before treatment was taken as 100% and contractions induced by EFS in the presence of the treatment were related to this reference value. In the acetylcholine release study, EFS evoked an increase in tritium overflow not only in samples 5 (S₁) and 25 (S₂) but also in up to maximally the 6 subsequent samples. Basal tritium overflow during the period with stimulation-induced increase of tritium overflow was calculated by fitting a regression line through the 4 samples just before stimulation and the 4 values starting from where overflow had returned to basal values after stimulation. The stimulation-induced increase in tritium overflow was then determined by subtracting basal tritium overflow from the values in the samples with increased overflow. The S₂/S₁ ratio was then calculated.

Results are expressed as means±SEM, n referring to tissues from different animals. Data obtained in parallel tissue groups were compared by an unpaired t-test (2 groups) or for more than 2 groups by ANOVA, followed by a post-hoc t-test corrected for multiple comparisons (Bonferroni). The influence of the increasing concentrations of the PDE-inhibitors on the electrically induced submaximal contractions was assessed by repeated measures ANOVA. P values of less than 0.05 were considered significant.

### Example 5

### Influence of 5-HT₄ Receptor Agonism on Cholinergic Nerve Endings

This example describes the influence of 5-HT₄ receptor agonism on cholinergic nerve endings, in particular at the effect of 5-HT₄ agonism on electrically-induced acetylcholine release from cholinergic nerve endings innervating pig gastric circular muscle. For this example, tritium outflow was considered a marker for acetylcholine release because changes in ³H-acetylcholine parallel changes in total tritium levels (See e.g. Leclere and Lefebvre, 2001).

Stimulation of cholinergic nerves in pig stomach muscle strips by EFS caused a clear-cut increase in tritium outflow above basal. The response induced by the second stimulation train was less pronounced yielding a S2/S1 ratio of 0.7 (Table 1). Incubation with prucalopride (0.03, 0.1 and 0.3 μM) prior to EFS, did not influence the basal outflow, however it significantly enhanced the tritium outflow induced by the second stimulation train leading to a concentration-dependent increase of the S2/S1 ratio with an S2/S1 ratio of 1.05 for 0.3 μM prucalopride (Table 1). In an additional series, the influence of 1 μM prucalopride was tested but this did not induce a more pronounced effect than 0.3 μM prucalopride (S2/S1 ratio: 0.74±0.05 for controls, n=5; 1.04±0.05 for 1 μM prucalopride, n=6; P<0.01).

TABLE 1
EFS-induced outflow of total radioactivity after incubation with prucalopride

| | | | | |
|---|---|---|---|---|
| S1 | 50952 ± 3496 | 68328 ± 11006 | 91698 ± 24563 | 61343 ± 11445 |
| Prucalopride (μM) | — (Control) | 0.03 | 0.1 | 0.3 |
| S2 | 35494 ± 3025 | 62398 ± 8272 | 91877 ± 21668 | 61498 ± 9813 |
| S2/S1 | 0.70 ± 0.04 | 0.97 ± 0.14 | 1.02 ± 0.03* | 1.05 ± 0.09* |

Total radioactivity (tritium) is expressed in dpm g⁻¹ tissue. For S1 and S2, the sum of radioactivity above baseline in sample 5 (S1) and sample 25 (S2), respectively, and the following samples with values above baseline is given. Means ± SEM of n = 5 to 6 tissues are given.
*P < 0.05 versus control without prucalopride.

The 5-HT₄ receptor antagonist GR 113808 (1, 10, 100 nM) did not influence basal tritium outflow but concentration-dependently antagonized the facilitating effect of 0.3 μM prucalopride, indicating that the effect of prucalopride on EFS-induced acetylcholine release is mediated via 5-HT4 receptors (Table 2).

TABLE 2
EFS-induced outflow of total radioactivity after
incubation with GR113808 followed by prucalopride

| | | | | |
|---|---|---|---|---|
| S1 | 50543 ± 3791 | 42314 ± 3744 | 45180 ± 10235 | 49850 ± 8210 |
| GR113808 (nM) | — (Control) | 1 | 10 | 100 |
| Prucalopride (μM) | 0.3 | 0.3 | 0.3 | 0.3 |
| S2 | 52591 ± 2950 | 43860 ± 4122 | 39273 ± 9533 | 47590 ± 8293 |
| S2/S1 | 1.05 ± 0.03 | 1.05 ± 0.08 | 0.86 ± 0.04 | 0.74 ± 0.05^{##} |

Total radioactivity (tritium) is expressed in dpm g⁻¹ tissue. For S1 and S2, the sum of radioactivity above baseline in sample 5 (S1) and sample 25 (S2), respectively, and the following samples with values above baseline is given. Means ± SEM of n = 5 to 6 tissues are given.
^{##}P < 0.01 versus control without addition of GR 113808 before prucalopride.

### Example 6

### Influence of Non-Selective PDE Inhibitors on the Effect of 5-HT₄ Receptor Agonists on Cholinergic Nerve Endings

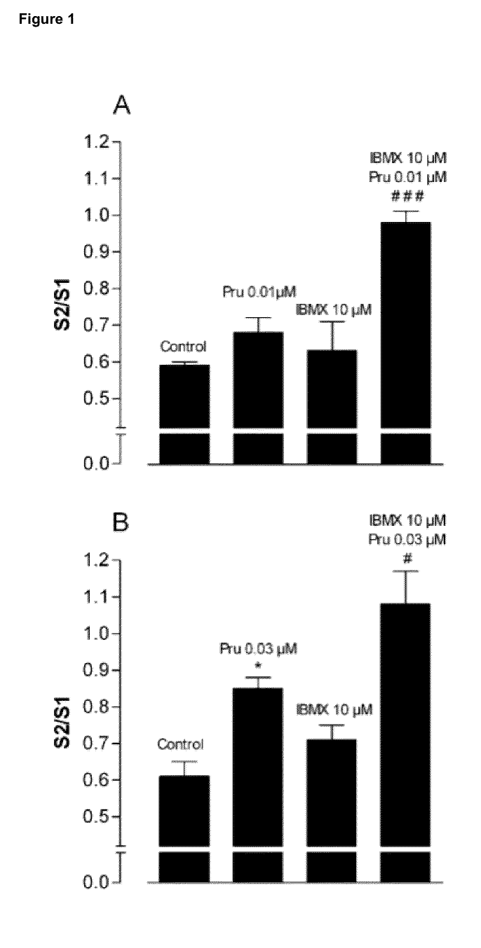

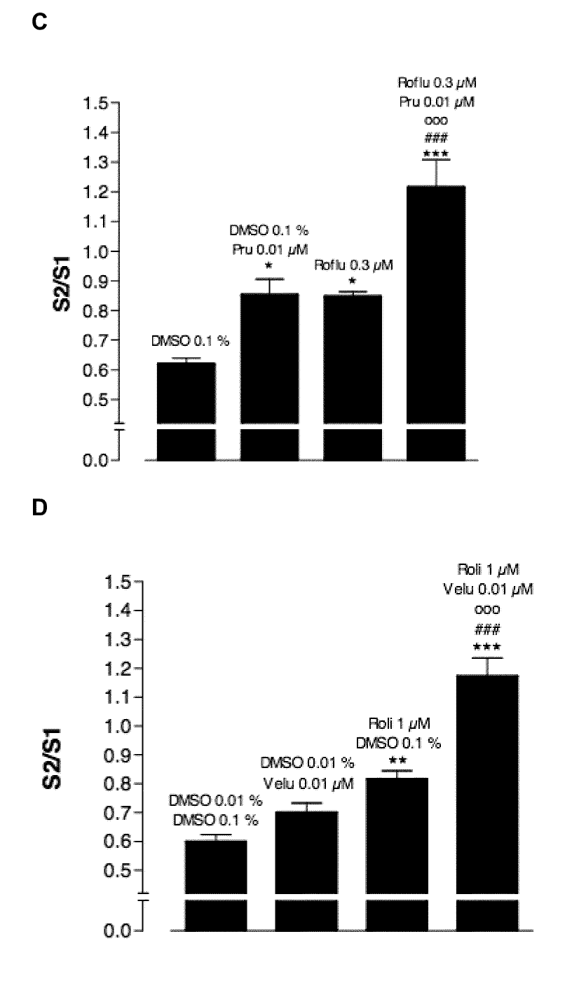

The influence of the non-specific PDE inhibitor IBMX (10 μM) was tested versus 0.01 μM prucalopride, a concentration that was minimally effective on acetylcholine release. Indeed, 0.01 μM prucalopride did not significantly increase EFS-induced tritium outflow versus control tissues: the S2/S1 ratio was not significantly different between tissues where 0.01 μM prucalopride was administered before S2 (0.68±0.04; n=6) versus that in control tissues (0.59±0.01; n=6) (FIG. 1A). IBMX (10 μM) per se did not influence basal nor did it influence EFS-induced tritium outflow (FIG. 1A). However, when IBMX was administered before prucalopride (0.01 μM), a clearcut significant increase in EFS-induced tritium outflow was obtained (FIG. 1A).

In a second series, 0.03 μM prucalopride alone enhanced EFS-induced tritium outflow (FIG. 1B). Again, IBMX (10 μM) alone did not significantly influence EFS-induced tritium outflow, however administration of IMBX before prucalopride, significantly increased tritium outflow compared to prucalopride alone (FIG. 1B).

### Example 7

### Influence of Selective PDE Inhibitors on the Effect of 5-HT₄ Receptor Agonists on Acetylcholine Release

### 7A

### Influence of Multiple Selective PDE Inhibitors on the Effect of Prucalopride on Acetylcholine Release

In this example, it was determined which of the PDE's was responsible for the observed facilitating effect of prucalopride on acetylcholine release by using multiple specific PDE inhibitors.

The PDE2 inhibitor EHNA (10 μM) did not influence basal nor EFS-induced tritium outflow. It also did not increase tritium outflow when administered in combination with 0.01 μM prucalopride compared to the tritium outflow attributable to prucalopride alone (S2/S1 ratio in control tissues: 0.53±0.02; with 10 μM EHNA: 0.51±0.05; with 0.01 μM prucalopride: 0.63±0.04; with EHNA and prucalopride: 0.58±0.03; n=4-6).

A small series of experiments was conducted wherein 0.01 μM prucalopride was added before S2, either alone or preceded by the PDE1 inhibitor vinpocetine (10 μM), the PDE3 inhibitor cilostamide (1 μM) or the PDE4 inhibitor rolipram (1 μM). None of these PDE-inhibitors alone influenced basal tritium outflow. However, the combination of the PDE4 inhibitor rolipram and prucalopride (S2/S1 ratio (0.98±0.02)) significantly enhanced EFS-induced tritium outflow (P<0.01) versus that in the presence of prucalopride alone (0.70±0.03; n=4). In contrast, neither the combination of the PDE1 inhibitor vinpocetine plus prucalopride (0.64±0.05; n=4) nor the combination of the PDE3 inhibitor cilostamide plus prucalopride (0.69±0.06; n=4) significantly increased EFS-induced tritium outflow when compared to prucalopride alone (0.70±0.03; n=4).

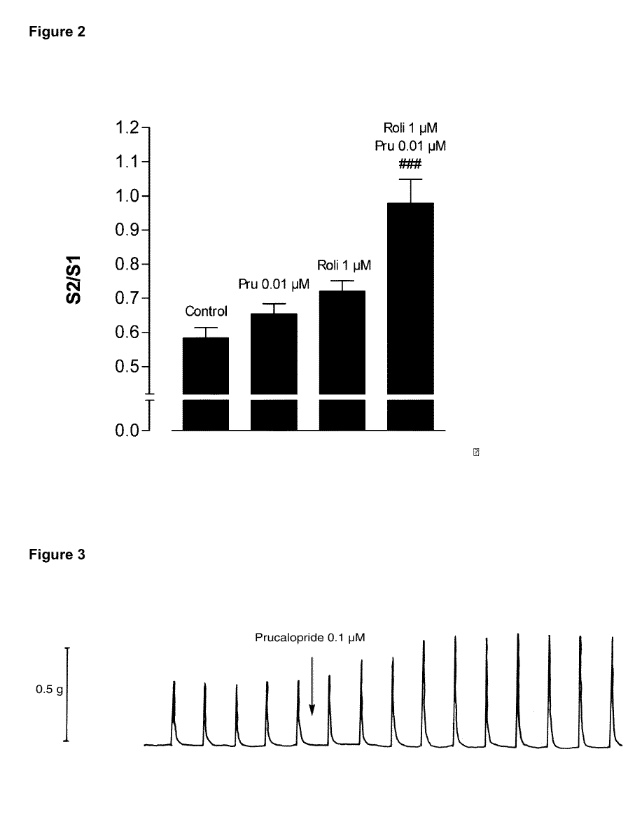

To further confirm the synergism between a 5-HT4 agonist and a PDE4 inhibitor, an additional series of experiments with the specific PDE4 inhibitor, rolipram, was also conducted. Rolipram (1 μM) alone increased the S2/S1 ratio but this was not significant compared to controls (FIG. 2). In contrast, the combination of rolipram and 0.01 μM prucalopride (0.98±0.07; n=6), significantly increased tritium outflow compared to prucalopride alone (0.65±0.03; n=6) (FIG. 2) yielding similar results as when using the combination of the non-selective PDE inhibitor IBMX and 0.01 μM prucalopride (FIG. 1A).

Our data show, that the specific PDE4 inhibitor rolipram in combination with prucalopride significantly increased EFS-induced tritium outflow, similarly as observed for the combination of IBMX with prucalopride.

### 7B

### Influence of Roflumilast (PDE4 Inhibitor) on the Effect of Prucalopride (5-HT₄ Receptor Agonist) on Cholinergic Acetylcholine Release

To further elaborate whether similar observations could be made with other selective PDE4 inhibitors, we further studied the influence of roflumilast on the effect of prucalopride in a similar setting.

The influence of 0.3 μM roflumilast, added from sample 13 onwards, was tested per se or versus 0.01 μM prucalopride, added from sample 20 onwards. In parallel tissues, the solvent of roflumilast (0.1% DMSO) was tested. Electrical stimulation induced an increase in tritium outflow in the sample with stimulation and the next two samples (Samples 5, 6 and 7 for S1 and samples 25, 26 and 27 for S2).

The mean S2/S1 ratios are shown in FIG. 1C. Prucalopride (0.01 μM) and roflumilast (0.3 μM) both evoked a moderate significant effect on the EFS-induced tritium outflow compared to control tissues. The S2/S1 ratio for prucalopride, added 15 min before S2, was 0.85±0.05 (n=6) and for roflumilast, added 36 min before S2, 0.85±0.02 (n=6) versus 0.62±0.02 (n=6) for the control strips.

When roflumilast (0.3 μM) was administered before prucalopride (0.01 μM), a clearcut significant increase in EFS-induced tritium outflow versus that in the presence of prucalopride alone or roflumilast alone was obtained (S2/S1 ratio of 1.22±0.09, n=6).

### 7C

### Influence of Rolipram (PDE4 Inhibitor) on the Effect of Velusetrag (5-HT₄ Receptor Agonist) on Acetylcholine Release

Where the foregoing study indeed shows that similar observations could be made with other selective PDE4 inhibitors, it was also determined whether similar observations can be made using other 5-HT₄ receptor agonist. Thus in this further study another 5-HT₄ receptor agonist has been used in a similar setting as for example 7A above.

The influence of 1 μM of the PDE4 inhibitor rolipram, added from sample 13 onwards, was tested per se or versus 0.01 μM velusetrag. The solvents of rolipram (0.01% DMSO) and velusetrag (0.1% DMSO) were taken in account.

The mean S2/S1 ratios are shown in FIG. 1D. The influence of rolipram was tested versus the 5HT4 receptor agonist velusetrag. Velusetrag (0.01 μM; S2/S1 ratio 0.7±0.03, n=6), added 15 min before S2, showed a minimal effect on EFS-induced tritium overflow versus control tissues (S2/S1 ratio 0.6±0.02, n=7).

Rolipram (1 μM), added 36 min before S2 significantly increased EFS-induced tritium outflow (S2/S1: 0.82±0.03, n=7). In the presence of rolipram and velusetrag, the S2/S1 ratio of total radioactivity outflow (1.17±0.06, n=7) was significantly enhanced compared to that in the presence of velusetrag alone or rolipram alone.

### Example 8

### Effect of 5-HT₄ Agonism on EFS-Induced Submaximal Cholinergic Contractions of Gastric Circular Muscles

Control circular muscle strips of the pig proximal stomach did not show spontaneous phasic activity and basal tone remained constant during the course of the experiment. Upon EFS induction, contractions at V50% C attained an amplitude of 67±10% (n=6) of that induced by 3 μM carbachol at the beginning of the experiment. These contractions were neurogenic and cholinergic as they were abolished by 3 μM tetrodotoxin (n=4) and 1 μM atropine (n=4) respectively. Upon repetitive stimulation, the amplitude of the EFS-induced contractions, in control tissue, at V50% C also remained stable. The amplitude of the contraction by a 15^{th} stimulation train was 100±5% of the mean response to trains 1 to 5; n=6.

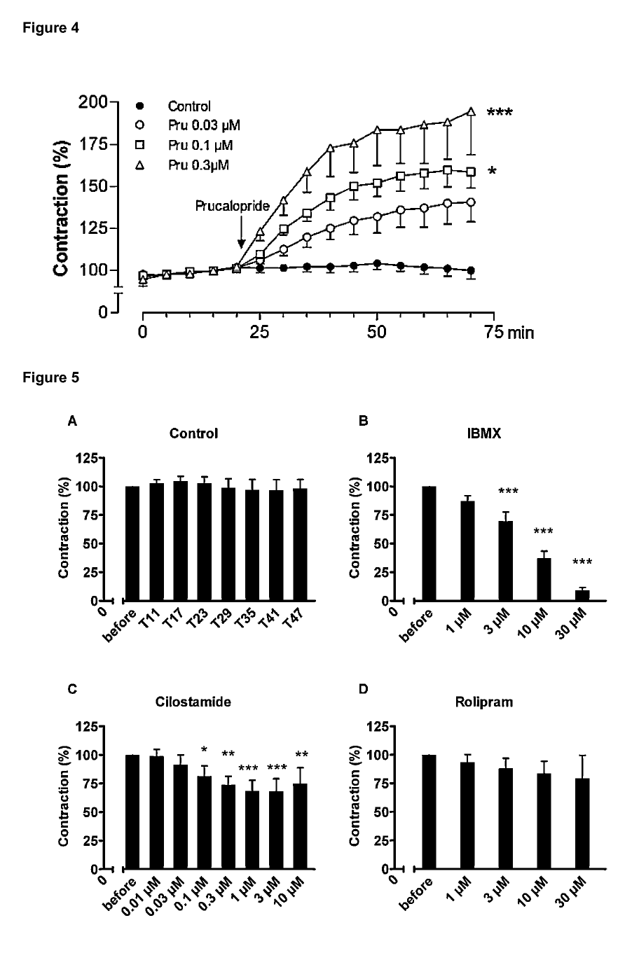

Incubation with the 5-HT₄ receptor agonist prucalopride, did not influence the basal tone of the strips, but it progressively enhanced the amplitude of the EFS-induced contractions (FIG. 3) coming close to the maximal effect for a given concentration at the 5^{th} stimulation train in its presence. The facilitating effect of prucalopride was concentration-dependent for the concentration range studied (0.03, 0.1 or 0.3 μM; FIG. 4).

The 5-HT4 receptor antagonist GR113808 (1, 10 and 100 nM) per se did not influence the EFS-induced contractions but concentration-dependently inhibited the facilitating effect of 0.3 μM prucalopride, demonstrating that the effect of prucalopride is mediated via activation of 5-HT4 receptors.

In conclusion, prucalopride progressively enhanced the amplitude of the EFS-induced cholinergic contractions, the facilitating effect being attenuated in the presence of a 5-HT4 receptor antagonist GR113808 indicating that regulation of electrically induced muscle contractions by prucalopride is due to its effect on acetylcholine release via 5-HT4 receptors.

### Example 9

### Influence of PDE Inhibitors on EFS-Induced Submaximal Cholinergic Contractions of Gastric Circular Muscles

A common problem associated with pharmaceutical drugs is their effect on multiple pathways and/or tissue types resulting in undesired side-effects. For example, it has been shown that 5-HT₄ stimulation in combination with non-selective inhibition of PDE (IBMX) or selective inhibition of PDE3 (cilostamide) whether or not in combination with selective inhibition of PDE4 (rolipram) increases the direct inotropic effect of 5-HT₄ stimulation on papillary muscles from post-infarction hearts (Afzal et al., 2008). As evident, in an attempt to provide an efficient way of increasing the prokinetic effect of 5-HT₄ receptor activation, it is undesired to have additional and direct effects on muscle tissue, which are not related to increased acetylcholine release.

In gastrointestinal smooth muscle, cyclic nucleotides such as cAMP are essential mediators of relaxation and their intracellular concentration is regulated by PDEs. The non-selective PDE-inhibitor IBMX induced a concentration-dependent reduction of the amplitude of the EFS-induced cholinergic contractions from 3 μM onwards, by functionally antagonizing the released acetylcholine at the muscular level (the contraction induced by acetylcholine is counteracted by a relaxation induced by increased cAMP levels in the smooth muscle cells). In the presence of 30 μM IBMX, the contractions were nearly abolished (FIG. 5B). None of the selective PDE-inhibitors was able to mimic the effect of IBMX. The PDE1-inhibitor vinpocetine (0.01-10 μM) and the PDE2-inhibitor EHNA (1-30 μM) did not significantly influence the submaximal cholinergic contractions (n=6 for each agent; data not shown), nor did the PDE4-inhibitor rolipram (1-30 μM; FIG. 5D). The PDE3-inhibitor cilostamide (0.01-10 μM) reduced the contractions from 0.1 μM onwards, however, the maximal depression obtained was much smaller than with IBMX (reduction to 68±11% with 3 μM cilostamide; FIG. 5C).

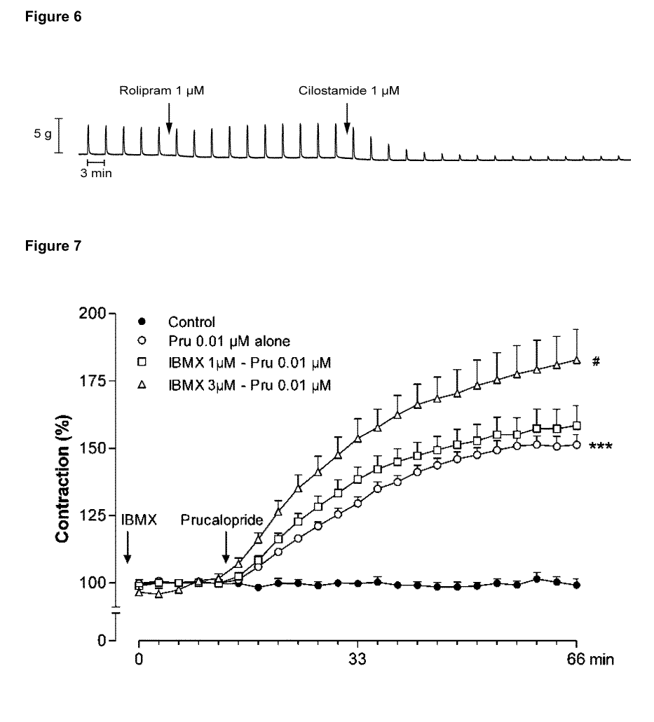

Sequential addition of the PDE3 inhibitor cilostamide (1 μM) after the PDE4 inhibitor rolipram (1 μM), substantially eliminated the electrically induced contractions (FIG. 6A). The response to the 10^{th} stimulation train in the combined presence of rolipram and cilostamide only attained 13±1% (n=4) of the response before adding the PDE-inhibitors. Also when the order of administration was reversed, electrically induced contractions were substantially eliminated. After first adding 1 μM cilostamide, the contraction decreased to 59±13% at the 10^{th} stimulation train in its presence; when further adding 1 μM rolipram, the contraction further decreased to 10±5% at the 10^{th} stimulation train in their combined presence.

In conclusion, none of the selective PDE inhibitors alone is able to substantially eliminate the electrically induced contractions to the same level as the non-selective PDE inhibitor IBMX. Only sequential addition of a PDE3 inhibitor and a PDE4 inhibitor obtained similar effects compared to IBMX. This indicates that both PDE3 and PDE4 are involved in regulating the concentrations of cAMP in smooth muscle cells of porcine gastric circular muscles and that a simultaneous inhibition of PDE3 and 4 is necessary to obtain a inhibitory effect on EFS-induced cholinergic contractions of gastric circular muscle. These data indicate that the PDE4 inhibitor, when not used in combination with PDE3, has no adverse effects on muscle contraction.

### Example 10

### Influence of PDE Inhibitors on the Effect of Prucalopride on EFS-Induced Submaximal Cholinergic Contractions of Gastric Circular Muscles

As shown in other examples, (see FIG. 5B) in gastric circular muscle strips of piglets, IBMX (1 and 3 μM), concentration-dependently decreased the EFS-induced contractions (maximally to 84±2%, n=6, in the presence of 3 μM IBMX). Therefore, to evaluate the effect of prucalopride, EFS-induced contractions in the presence of prucalopride were expressed as % of the mean of the last 5 EFS-induced contractions in the presence of IBMX just before adding prucalopride (FIG. 7). This showed a significant enhancement of the facilitating effect of prucalopride by 3 μM IBMX in comparison to prucalopride alone (FIG. 7). In an additional series, the influence of 10 μM IBMX was studied. When added in the presence of 10 μM IBMX, the enhancement was more pronounced than for prucalopride alone, although this did not reach significance (data not shown). These data indicate that a non-specific PDE-inhibitor enhances the facilitating effect of prucalopride on ESF induced, i.e. on cholinergic contractions of gastric muscle cells. Based on the results of the previous experiments that specific inhibition of PDE4 synergistically enhances the facilitating effect of prucalopride on acetylcholine release from cholinergic nerve endings (See FIG. 2), we further tested whether PDE4 inhibition was responsible for the enhancement of the facilitating effect of prucalopride on EFS induced contractions by IBMX.

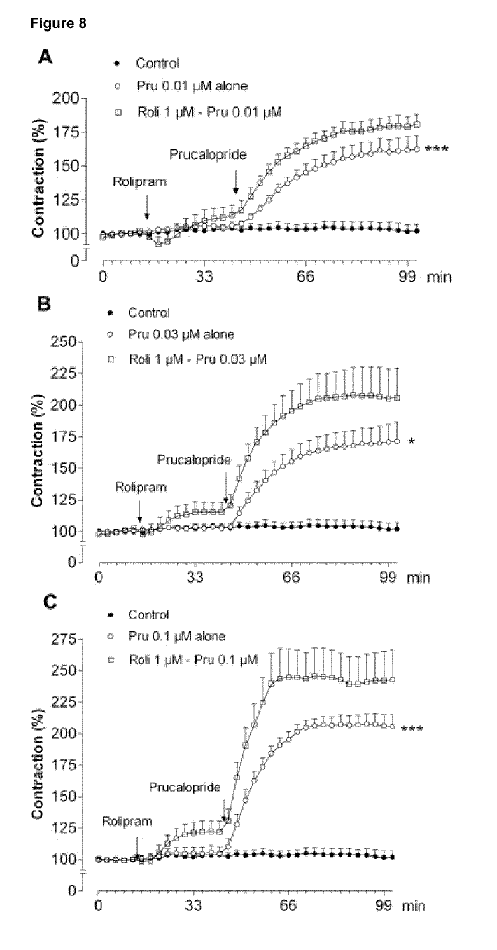

Rolipram (1 μM) was tested versus 0.01, 0.03 and 0.1 μM prucalopride (FIG. 8). In this series, the mean contractile response to the 10^{th} stimulation train in the presence of rolipram was somewhat increased in comparison to the response before its administration to:
114±8% (n=8) before 0.01 μM prucalopride (FIG. 8A)
115±8% (n=8) before 0.03 μM prucalopride (FIG. 8B)
122±9% (n=8) before 0.1 μM prucalopride (FIG. 8C)

This was due to an increase in the response to stimulation in the presence of rolipram in some tissues. For example, in the tissues where 0.03 μM prucalopride was going to be added, the individual contractile response to the 10^{th} stimulation in the presence of rolipram was 96, 111, 137, 155, 93, 102, 101 and 128%.

Prucalopride alone increased the electrically induced contractions to:
162±11% (n=7; 0.01 μM; FIG. 8A)
171±15% (n=8; 0.03 μM; FIG. 8B)
206±10% (n=7; 0.1 μM; FIG. 8C)

When rolipram had been added before prucalopride, the combination increased the electrically induced concentrations:
181±7% (n=8: 0.01 μM)—FIG. 8A
206±24% (n=8; 0.03 μM)—FIG. 8B
243±23% (n=8; 0.1 μM)—FIG. 8C

In conclusion, also at the level of EFS-induced submaximal cholinergic contractions of gastric circular muscles, the specific PDE4 inhibitor mimics the behavior of the non-specific PDE inhibitor IBMX. However, contrary to the specific PDE4 inhibitor, the non-specific PDE inhibitor IBMX has an undesired inhibiting effect on gastric muscle contraction (see FIG. 5B).

We have now clearly shown a synergistic result of the facilitating effect of prucalopride on cholinergic acetylcholine release and cholinergic gastric muscle contractions when in combination with a specific inhibition of PDE4. Furthermore, as PDE4 inhibition on its own has no inhibiting effect on smooth circular muscles, including gastric circular muscles, the combination of PDE4 inhibitor with 5-HT₄ receptor antagonism is a way of synergistically enhancing the facilitating effect of prucalopride by specifically targeting the cholinergic neurotransmission and acetylcholine release when in combination with a PDE4 inhibitor.

### Part B: Colonic Circular Muscles Experiments

This part of the study shows the results for colonic tissue using smooth muscle strips of the colon of a test animal.

### Example 11

### Preparation of Smooth Muscle Strips of the Colon of a Test Animal

Young male pigs (10-12 weeks, 15-25 kg-breed Line 36) were obtained from Rattlerow Seghers, Belgium. On the morning of the experiment, pigs were anaesthetized with an intramuscular injection of 5 ml Zoletil 100 (containing 50 mg/ml tiletamine and 50 mg/ml zolazepam; Virbac Belgium S.A., Belgium). After exsanguination, the colon descendens was prelevated 10 cm above the anus to the transverse colon and was placed in aerated (5% CO₂/95% O₂) Krebs-Henseleit solution (composition in mM: glucose 11.1, NaHCO₃ 25, KHPO₄ 1.18, CaCl₂ 2.51, MgSO₄ 1.18, KCl 4.69, NaCl 118).

For preparation of the smooth muscle strips, the colon descendents was opened along the mesenteric border and after removal of the mucosa, 8 full-thickness circular muscle strips (approx. 3×20 mm) were prepared in pairs at the same level, starting 2 cm above the distal end. The strips were mounted in 10 ml organ baths between 2 platinum plate electrodes under a load of 2 g to allow electrical field stimulation (EFS) performed by means of a 4 channel custom-made stimulator.

### Example 12

### Methodology for Studying the Electrically-Induced Contractions of Colon Muscles

The aerated (5% CO₂/95% O₂) Krebs-Henseleit solution in the organ baths (see example 11) systematically contained 4 μM of the noradrenergic neuron blocker guanethidine and 0.3 mM of the NO synthase inhibitor N_{ω}-nitro-L-arginine methyl ester hydrochloride (L-NAME) to avoid noradrenergic and nitrergic responses respectively.

After 60 min of stabilization with refreshing of the Krebs-Henseleit solution every 15 min, strips were contracted with the muscarinic receptor agonist carbachol (3 μM). This procedure was repeated with a 20-min washout period in between. After the second carbachol administration and washout period, the small conductance calcium-dependent potassium channel blocker apamin (0.5 μM) and a combination of the tachykinin receptor antagonists (NK₁, 10 μM FK888; NK₂,1 μM MEN10627; NK₃, 0.3 μM SB222200) were added and incubated for 30 min before the first electrical stimulation. We previously showed that the addition of the tachykinin receptor antagonists to the medium, also containing guanethidine, L-NAME and apamin allows to obtain reproducible cholinergic contractions by EFS (Priem and Lefebvre, 2011).

Strips were then stimulated for 1 hour (12 stimulations) with 5 min interval at supramaximal voltage (35 V) (10 s trains; 0.25 ms pulse duration; frequency of 4 Hz). After hour, EFS was stopped, muscle strips were rinsed and apamin (0.5 μM) and the combination of the tachykinin receptor antagonists was again added and incubated for 30 min before the next stimulation. EFS (10 s; 0.25 ms; 4 Hz) was then applied with 5 min interval at an initial voltage of 15 V. The voltage was further adjusted to reduce the contraction force to approximately 50% (V50%) of the force evoked at 35 V and EFS was repeated until 5 reproducible contractions were obtained at V50%. The protocols as described in examples 12 and 13 then started. Experiments where the EFS-induced submaximal contractions in time controls decreased by more than 25% in the course of the experiment, were not taken in account (14/48).

Changes in isometric tension were measured using MLT 050/D force transducers (ADInstruments, United Kingdom) and recorded on a PowerLab/8sp data recording system (ADInstruments, United Kingdom) with Chart v5.5.6 software.

The obtained data were analysed as follows: Stimulation trains were numbered starting from the 5 consecutive stimulations at V50% with reproducible contractions just before adding substances (1, 2, 3, 4, 5, . . . ). The mean contractile response to these 5 stimulations was taken as 100% reference for all the following responses.

Results are expressed as means±S.E.M., n referring to tissues from different animals except when otherwise indicated. Statistical analysis was performed by use of Graphpad Prism v.5.01 (San Diego, U.S.A.); P<0.05 was considered statistically significant. When adding PDE inhibitors cumulatively, the last contraction in the presence of each concentration was compared to the reference by repeated measures ANOVA followed by a Bonferroni corrected t-test. In experiments, where prucalopride was added after a PDE inhibitor, responses induced by stimulation 13, corresponding to the 2^{nd} stimulation after adding prucalopride, were compared between the time controls, the tissues with prucalopride alone and the tissues with addition of prucalopride after a PDE inhibitor was added, by ONE-WAY ANOVA followed by a Bonferroni corrected t-test. In the experiments, where rolipram was added after prucalopride, the response to stimulation 7 (i.e. the 2^{nd} stimulation after adding prucalopride) was compared to the mean response to stimulations 3 to 5 by a paired t-test; the response by stimulation 19 (i.e. the 2^{nd} stimulation after adding rolipram) was similarly compared to the mean response to stimulations 15 to 17.

### Example 13

### Influence of PDE Inhibitors per se on EFS-Induced Submaximal Cholinergic Contractions of Colon Circular Muscles

The influence of the non-selective PDE inhibitor 3-isobutyl-1-methyl-xanthine (IBMX) and the selective PDE inhibitors vinpocetine (PDE1 inhibitor), EHNA (PDE2 inhibitor), cilostamide (PDE3 inhibitor), rolipram (PDE4 inhibitor) and zaprinast (PDE5 inhibitor) was tested on EFS-evoked submaximal (V50%) cholinergic contractions. A cumulative concentration-response curve for the different PDE inhibitors was obtained by adding them in half log unit increasing concentrations, starting after 5 reproducible contractions at V50% had been obtained and registering the responses to 6 trains (30 min) after adding each concentration. Parallel to the cumulative concentration-response curve of rolipram, an isolated concentration-response curve was obtained by adding one single concentration per tissue in 3 animals. Control tissues did not receive any solvent nor PDE inhibitor. The solvents DMSO and ethanol were tested separately by adding them cumulatively in the matching dilutions as for the cumulative concentration series of the corresponding PDE inhibitor.

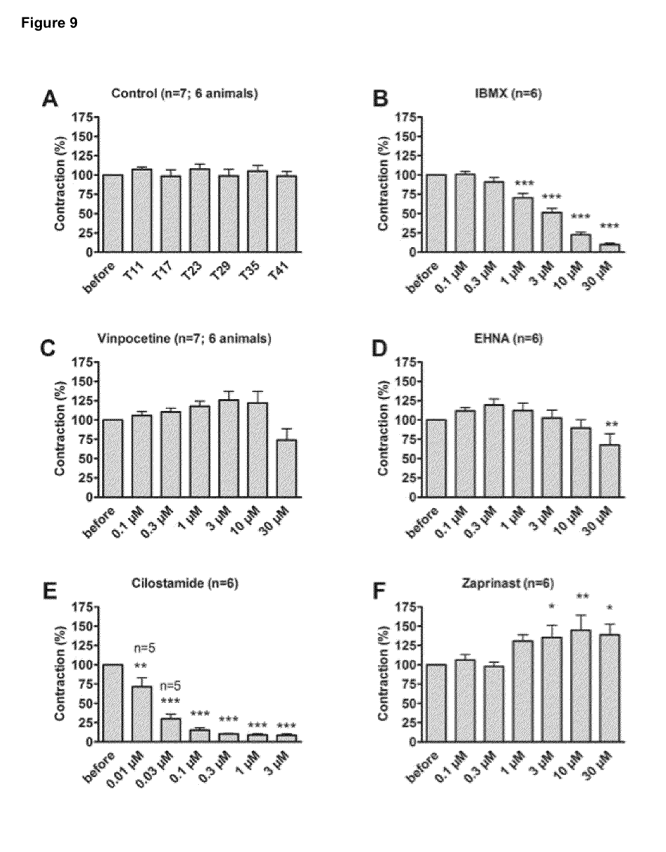

In the control tissues shown in FIG. 9A, the contractile response by EFS at supramaximal voltage (35 V) was 43±5% (n=7; 6 animals) of that induced by 3 μM carbachol at the beginning of the experiment. Once stimulation voltage was reduced to V50%, EFS-induced contractions in these control tissues attained an amplitude of 52±3% (n=7; 6 animals) of that induced at supramaximal voltage of 35 V. In the control tissues, the amplitude of the contractile responses by EFS at V50% remained stable upon repetitive stimulation (amplitude of the contraction at the last stimulation was 94±6% of the mean response to stimulation train 1 to 5 (n=7; 6 animals).

Two PDE inhibitors concentration-dependently inhibited EFS-induced cholinergic contractions in circular muscle of pig colon descendens: IBMX (FIG. 9B) and the PDE3 selective inhibitor cilostamide (FIG. 9E). The concentration range where IBMX showed its concentration-dependent effect (1-30 μM) corresponds to the IC₅₀ range of this non selective PDE inhibitor (2-50 μM; Beavo and Reifsnyder, 1990). None of the PDE subtype selective inhibitors (FIG. 9C-F) mimicked the inhibitory effect of IBMX except for cilostamide (FIG. 9E), being about 100 times more potent than IBMX. Reported IC₅₀ values for cilostamide at PDE3 include 0.005 and 0.064 μM (Elks and Manganiello, 1984; Beavo and Reifsnyder, 1990). In this concentration range (0.03 μM), cilostamide already inhibited EFS-induced cholinergic contractions by 75%.

These results illustrate that PDE3 is key in controlling cyclic nucleotide levels in colon descendens circular muscle, and that the use of a PDE3 inhibitor has counteracting effect on muscle contraction, as shown by the inhibitory effect on EFS-induced cholinergic contractions. In contrast, and in analogy with the observations on gastric muscle, also on colonic muscle PDE4 inhibitors do not cause a relaxation of the GI smooth muscles.

The principal role of PDE3 in pig colon descendens circular muscle differs from the results in pig gastric circular muscle (see part A of the examples), where we observed a redundant role of PDE3 and PDE4 in controlling cyclic nucleotide levels with PDE3 being predominant.

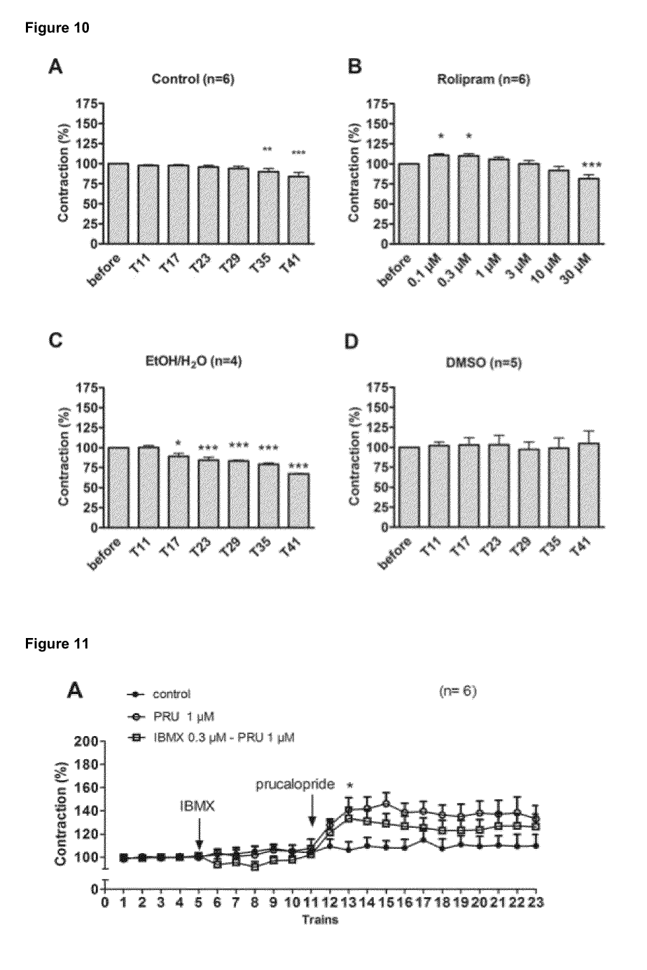

A significant increase of the EFS-induced contractions in pig colon was also seen with 0.1 and 0.3 μM of the PDE4 inhibitor rolipram (FIG. 10 B). Also in pig gastric muscle (see part A of the examples), rolipram tended to increase electrically induced acetylcholine release and cholinergic contraction, suggesting some basal control by PDE4 of acetylcholine release per se from cholinergic nerves

### Example 14

### Influence of PDE Inhibitors on the Effect of 5-HT₄ Agonists on EFS-Induced Submaximal Cholinergic Contractions in the Colon

In porcine left atrium, the 5-HT₄ receptor is under very tight control of PDE3 and PDE4, as prucalopride only has a very moderate and fading effect in the absence of both PDE3 and PDE4 inhibitors (De Maeyer et al., 2006b; Galindo-Tovar et al., 2009; Weninger et al., 2012). We therefore tested the influence of inhibitors of the PDEs that metabolize cAMP on the response to prucalopride in pig colon descendens, except for the PDE3 inhibitor cilostamide in view of its pronounced effect at the level of the muscle cells. Similar to pig gastric circular muscle, the PDE1 inhibitor vinpocetine (data not shown) and the PDE2 inhibitor EHNA (data not shown) did not influence the facilitating effect of prucalopride on cholinergic neurotransmission.

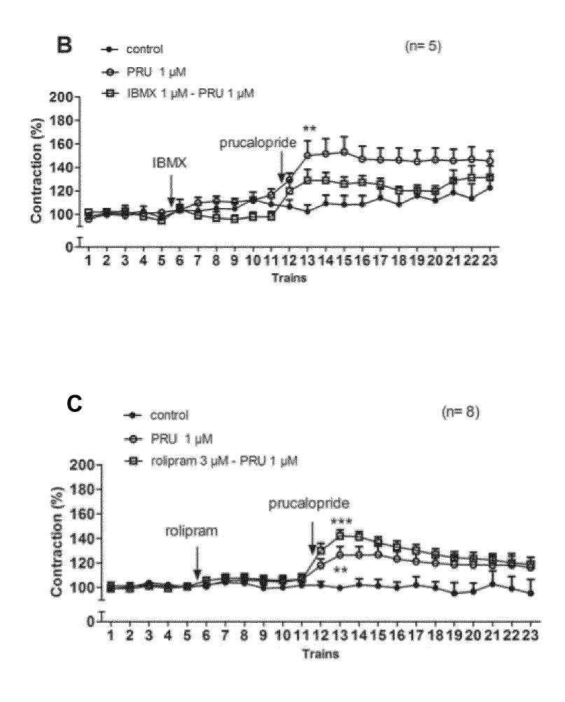

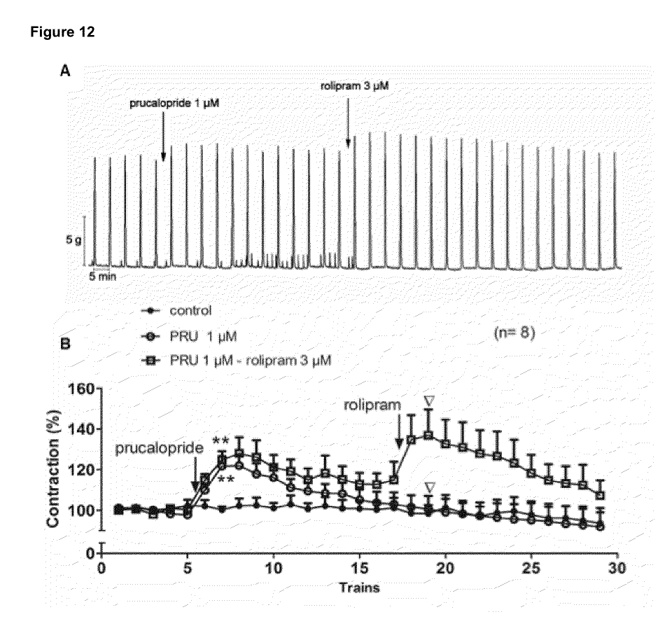

The selective 5-HT₄ receptor agonist prucalopride (1 μM) systematically enhanced EFS-induced cholinergic submaximal contractions, confirming the presence of facilitating 5-HT₄ receptors on the cholinergic nerve endings in pig colon descendens circular muscle (Priem and Lefebvre, 2011). When rolipram, 3 μM, was administered before prucalopride, it did not enhanced the EFS-induced contractions (FIG. 11C) but the EFS-induced contractions after adding prucalopride attained higher values than with prucalopride alone. Furthermore, when 3 μM rolipram was added after prucalopride (FIG. 12), it induced a clearcut and significant enhancement of the EFS-induced responses (FIGS. 12 A and B). This confirms in the colon what has also been found in gastric tissue (see example 10), i.e. an enhancement of cholinergic neurotransmission when combining a 5-HT4 receptor agonist and PDE4 inhibitor.

### REFERENCES

Afzal F, Andressen K W, Mork H K, Aronsen J M, Sjaastad I, Dahl C P, Skomedal T, Levy F O, Osnes J B, Qvigstad E., 2008. 5-HT4-elicited positive inotropic response is mediated by cAMP and regulated by PDE3 in failing rat and human cardiac ventricles. Br J. Pharmacol. 155: 1005-14.
Barbier, A. J., Lefebvre, R. A., 1995. Relaxant influence of phosphodiesterase inhibitors in the cat gastric fundus. Eur. J. Pharmacol. 276, 41-47
Barnette, M. S., Manning, C. D., Price, W. J., Barone, F. C., 1993. Initial biochemical and functional characterization of cyclic nucleotide phosphodiesterase isozymes in canine colonic smooth muscle. J Pharmacol Exp Ther. 264(2), 801-812
Beattie D T, Armstrong S R, Vickery R G, Tsuruda P R, Campbell C B, Richardson C, McCullough J L, Daniels O, Kersey K, Li Y-P and Kim K H S. The pharmacology of TD-8954, a potent and selective 5-HT4 receptor agonist with gastrointestinal prokinetic properties. Frontiers in Pharmacol 2011, 2, article 25, 1-13.
Beavo, J. A., Reifsnyder, D. H., 1990. Primary sequence of cyclic nucleotide phosphodiesterase isozymes and the design of selective inhibitors. Trends. Pharmacol. Sci. 11, 150-155
De Maeyer J H, Lefebvre R A, Schuurkes J A., 2008. 5-HT4 receptor agonists: similar but not the same. Neurogastroenterol Motil. 20: 99-112.
De Maeyer J H, Prins N H, Schuurkes J A J and Lefebvre R A. Differential effects of 5-hydroxytryptamine-4 receptor agonists at gastric versus cardiac receptors: An operational framework to explain and quantify organ-specific behavior. J Pharmacol Exp Ther 2006, 317, 955-964.
De Maeyer, J. H., Straetemans, R., Schuurkes, J. A., Lefebvre R A., 2006b. Porcine left atrial and sinoatrial 5-HT(4) receptor-induced responses: fading of the response and influence of development. Br J. Pharmacol. 147, 140-157
Elks, M. L., Manganiello, V. C., 1984. Selective effects of phosphodiesterase inhibitors on different phosphodiesterases, adenosine 3′,5′-monophosphate metabolism, and lipolysis in 3T3-L1 adipocytes. Endocrinology 115, 1262-1268
Galindo-Tovar, A., Vargas, M. L., Escudero, E., Kaumann, A. J., 2009. Ontogenic changes of the control by phosphodiesterase-3 and -4 of 5-HT responses in porcine heart and relevance to human atrial 5-HT(4) receptors. Br J. Pharmacol. 156, 237-249
Hebeiss, K., Kilbinger, H., 1996. Differential effects of nitric oxide donors on basal and electrically evoked release of acetylcholine from guinea-pig myenteric neurones. Br. J. Pharmacol. 118, 2073-2078
Jones, O. M., Brading, A. F., McC Mortensen, N. J., 2002. Phosphodiesterase inhibitors cause relaxation of the internal anal sphincter in vitro. Dis. Colon Rectum. 45, 530-536
Kaneda, T., Shimizu, K., Nakajyo, S., Urakawa, N., 1997. Effects of various selective phosphodiesterase inhibitors on muscle contractility in guinea pig ileal longitudinal smooth muscle. Jpn. J. Pharmacol. 75, 77-85
Kaumann A J, Lynham J A, Brown A M., 1996. Comparison of the densities of 5-HT4 receptors, beta 1- and beta 2-adrenoceptors in human atrium: functional implications. Naunyn Schmiedebergs Arch Pharmacol. 353: 592-5
Langlois M, Fischmeister R., 2003. 5-HT4 receptor ligands: applications and new prospects. J Med. Chem. 46: 319-44
Leclere, P. G., Lefebvre, R. A., 2001. Influence of nitric oxide donors and of the α2-agonist UK-14,304 on acetylcholine release in the pig gastric fundus. Neuropharmacol. 40, 270-278.
Maurice, D. H., Palmer, D., Tilley, D. G., Dunkerley, H. A., Netherton, S. J., Raymond, D. R., Elbatarny, H. S., Jimmo, S. L., 2003. Cyclic nucleotide phosphodiesterase activity, expression, and targeting in cells of the cardiovascular system. Mol. Pharmacol. 64, 533-546
Murthy, K. S., 2001. Activation of phosphodiesterase 5 and inhibition of guanylate cyclase by cGMP-dependent protein kinase in smooth muscle. Biochem. J. 360, 199-208
Murthy, K. S., Zhou, H., Makhlouf, G. M., 2002. PKA-dependent activation of PDE3A and PDE4 and inhibition of adenylyl cyclase V/VI in smooth muscle. Am. J. Physiol. Cell. Physiol. 282, C508-C517
Priem, E. K. V., Lefebvre, R. A., 2011. Investigation of neurogenic excitatory and inhibitory motor responses and their control by 5-HT4 receptors in circular smooth muscle of pig descending colon. Eur. J. Pharm. 667, 365-374
Priem, E., Van Colen, I., De Maeyer, J. H., Lefebvre, R. A., 2012. The facilitating effect of prucalopride on cholinergic neurotransmission in pig gastric muscle is regulated by phosphodiesterase 4. Neuropharmacology 62, 2126-2135
Reese, J. H., Cooper, J. R., 1984. Stimulation of acetylcholine release from guinea-pig ileal synaptosomes by cyclic nucleotides and forskolin. Biochem. Pharmacol. 33, 3007-3011
Stephenson, D. T., Coskran, T. M., Wilhelms, M. B.,
Adamowicz, W. O., O'Donnell, M. M., Muravnick, K. B., Menniti, F. S., Kleiman, R. J., Morton, D., 2009. Immunohistochemical localization of phosphodiesterase 2A in multiple mammalian species. J. Histochem. Cytochem. 57, 933-949
Tomkinson, A., Raeburn, D., 1996. The effect of isoenzyme-selective PDE inhibitors on methacholine-induced contraction of guinea-pig and rat ileum. Br. J. Pharmacol. 118, 2131-2139
Ward, S. M., Dalziel, H. H., Bradley, M. E., Buxton, I. L., Keef, K., Westfall, D. P., Sanders, K. M., 1992. Involvement of cyclic GMP in non-adrenergic, non-cholinergic inhibitory neurotransmission in dog proximal colon. Br. J. Pharmacol. 107, 1075-1082
Weninger, S., De Maeyer, J. H., Lefebvre, R. A., 2012. Study of the regulation of the inotropic response to 5-HT(4) receptor activation via phosphodiesterases and its cross-talk with C-type natriuretic peptide in porcine left atrium. Naunyn Schmiedebergs Arch. Pharmacol. 385, 565-577
Wohlsen A, Hirrle A, Tenor H, Marx D and Beume R. Effect of cyclic AMP-elevating agents on airway ciliary beat frequency in central and lateral airways in rat precision-cut lung slices. Eur J Pharmacol 2010, 635, 177-183.
Yau, W. M., Dorsett, J. A., Youther, M. L., 1987. Stimulation of acetylcholine release from myenteric neurons of guinea pig small intestine by forskolin and cyclic AMP. J. Pharmacol. Exp. Ther. 243, 507-510
Zhang, X. Y., Robinson, N. E., Zhu, F. X., 1996. Potentiation of acetylcholine release from tracheal parasympathetic nerves by cAMP. Am. J. Physiol. 270, L541-L546

1. A combination of at least one 5-HT₄ receptor agonist and at least one phosphodiesterase 4 (PDE4) inhibitor.

2. The combination according to claim 1, wherein the 5-HT₄ receptor agonist is selected from the group consisting of prucalopride, cisapride, mosapride, renzapride, naronapride, zacopride, tegaserod, dazopride, velusetrag, metoclopramide, cinitapride, YM-53389{(+)-(S)-2-chloro-5-methoxy-4-[5-(2-piperidylmethyl)-1,2,4-oxadiazol-3-yl]aniline monohydrochloride}, RS-67333, 5-Methoxytryptamine (5-MT), and BIMU-8.

3. The combination according to claim 1, wherein the phosphodiesterase 4 (PDE4) inhibitor is selected from the group consisting of rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, Mesembrine, Ro20-1724, RPL-554, and YM-976.

4. The combination according to claim 1, wherein the 5-HT₄ receptor agonist is prucalopride and the PDE4 inhibitor is roflumilast.

5. The combination according to claim 1, wherein acetylcholine is released from cholinergic neurons innervating gastric and/or colonic circular muscle cells when the composition is administered to a patient.

6. The combination of claim 5, wherein the amount of acetylcholine released is greater than levels of acetycholine after individual exposure to a 5-HT₄ receptor agonist and a PDE4 inhibitor under the same conditions and for the same time.

7. A method of stimulating the release of acetylcholine from cholinergic neurons innervating gastric and/or colonic circular muscle cells comprising exposing for a sufficient time the cells to a combination comprising a pharmaceutically acceptable amount of at least one 5-HT₄ receptor agonist and at least one phosphodiesterase 4 (PDE4) inhibitor.

8. The method of claim 7, wherein exposing the cells to the combination results in a level of acetylcholine that is greater than levels of acetycholine after individual exposure to a 5-HT₄ receptor agonist and a PDE4 inhibitor under the same conditions and for the same period of time.

9. The method of claim 7, wherein acetylcholine release is associated with prevention and/or treatment of a disorder selected from the group consisting of gastrointestinal, urinary, and respiratory disorders.

10. The method of claim 9, wherein the gastrointestinal disorder is selected from the group consisting of irritable bowel syndrome, chronic constipation, constipation caused by spinal cord injury or pelvic diaphragm failure, intestinal atony, reflux esophagitis, gastroesophageal reflux disorder (GERD), Barrett syndrome, intestinal pseudoileus, acute or chronic gastritis, gastric or duodenal ulcer, Crohn's disease, non-ulcer dyspepsia, gastroparesis, functional dyspepsia, ulcerative colitis, postgastrectomy syndrome, postoperative digestive function failure, delayed gastric emptying caused by gastric neurosis, and indigestion.

11. A method of treating a gastrointestional disorder, urinary disorder or respiratory disorder in a patient suffering therefrom comprising administering to the patient an effective amount of a combination comprising at least one 5-HT₄ receptor agonist and at least one phosphodiesterase 4 (PDE4) inhibitor.

12. The method of claim 11, wherein the gastrointestinal disorder is selected from the group consisting of irritable bowel syndrome, chronic constipation, constipation caused by spinal cord injury or pelvic diaphragm failure, intestinal atony, reflux esophagitis, gastroesophageal reflux disorder (GERD), Barrett syndrome, intestinal pseudoileus, acute or chronic gastritis, gastric or duodenal ulcer, Crohn's disease, non-ulcer dyspepsia, gastroparesis, functional dyspepsia, ulcerative colitis, postgastrectomy syndrome, postoperative digestive function failure, delayed gastric emptying caused by gastric neurosis, and indigestion.

13. The method of claim 11, wherein the 5-HT₄ receptor agonist is selected from the group consisting of prucalopride, cisapride, mosapride, renzapride, naronapride, zacopride, tegaserod, dazopride, velusetrag, metoclopramide, cinitapride, YM-53389{(+)-(S)-2-chloro-5-methoxy-4-[5-(2-piperidylmethyl)-1,2,4-oxadiazol-3-yl]aniline monohydrochloride}, RS-67333, 5-Methoxytrytamine (5-MT), and BIMU-8.

14. The method of claim 11, wherein the phosphodiesterase (PDE4) inhibitor is selected from the group consisting of rolipram, mesembrine, drotaverine, roflumilast, ibudilast, piclamilast, luteolin, cilomilast, diazepam, arofylline, CP-80633, denbutylline, drotaverine, etazolate, filaminast, glaucine, HT-0712, ICI-63197, irsogladine, Mesembrine, Ro20-1724, RPL-554, and YM-976.

15. The method of claim 11, wherein the 5-HT₄ receptor agonist is prucalopride and the PDE4 inhibitor is roflumilast.

16. The method of claim 11, wherein the step of administering to the patient the effective amount of the combination comprising the 5-HT₄ receptor agonist and the phosphodiesterase (PDE4) inhibitor selectively releases acetylcholine from cholinergic neurons innervating gastric and/or colonic circular muscle cells.

17. A method of selectively stimulating gastric and/or colonic smooth muscle cell contraction comprising exposing cholinergic neurons innervating the cell with an effective amount of a combination comprising a 5-HT₄ receptor agonist and a phosphodiesterase (PDE4) inhibitor, and releasing acetylcholine from cholinergic neurons towards the cell to stimulate contraction, wherein substantially no cAMP-mediated smooth muscle relaxation and/or atrial muscle contraction occurs.
